(19) United States
(12) Patent Application Publication
(10) Pub. No.: US 2022/0080020 A1
(43) Pub. Date: Mar. 17, 2022

(54) COMPOSITIONS AND METHODS FOR REDUCING THE TRANSMISSIVITY OF ILLNESSES USING AN ORAL DELIVERY SYSTEM

(71) Applicants: Luc Montagnier, New York, NY (US); Lee H. Lorenzen, Ranch Santa Margarita, CA (US)

(72) Inventors: Luc Montagnier, New York, NY (US); Lee H. Lorenzen, Ranch Santa Margarita, CA (US)

(21) Appl. No.: 17/409,669

(22) Filed: Aug. 23, 2021

Related U.S. Application Data
(60) Provisional application No. 63/068,994, filed on Aug. 22, 2020.

(51) Int. Cl. A61K 36/9066; A61K 31/704; A61K 31/352; A61K 33/38; A61K 31/047; A61K 31/375; A61K 36/752; A61K 31/23; A61K 31/122; A61K 33/30; A61K 31/4709; A61K 9/14; A61K 47/10; A61K 9/08

(52) U.S. Cl. CPC A61K 36/9066; A61K 31/704; A61K 31/352; A61K 33/38; A61K 31/047; A61K 31/375; A61K 9/08; A61K 31/23; A61K 31/122; A61K 33/30; A61K 31/4709; A61K 9/14; A61K 47/10; A61K 36/752

(57) ABSTRACT

Methods for prophylactic and anti-transmissivity uses of an anti-microbial composition are disclosed. The methods comprise the step of administering to a human an amount of a composition having a first ingredient being xylitol; a second ingredient comprised of a glycyrrhizin-protargin-quercetin complex, in addition to formula stabilizing ingredients of glycerol monolaurate, grapefruit seed extract; vitamin C (ascorbic acid), aloe emodin, curcumin, 1,8-cineole, zinc, quinine, flavoring, and an acceptable preservative for use in an oral application. When administered the composition is effective in reducing the incidence of contracting an illness or to prophylactically help prevent transmission of an illness into the or cavity and respiratory tract.

### CROSS REFERENCE TO RELATED APPLICATIONS

The present application is a Non-Provisional of, and claims benefit of priority under 35 U.S.C. § 119(e) from, U.S. Provisional Application No. 63/068,994, filed Aug. 22, 2020, the entirety of which is expressly incorporated herein by reference.

### FIELD OF THE INVENTION

The present invention relates to the field of antiviral and antibacterial medicaments, and more particularly to a silver composition for preventative and therapeutic application.

### BACKGROUND OF THE INVENTION

Viral pathogenesis is the method by which viruses produce disease in the host. The pathogenesis of viruses centers on the mechanisms of viral injury to discrete populations of cells in particular organs to produce signs and symptoms of disease in a particular host.

To initiate an infection the virus must gain entry to the host cell. Entry routes are dependent on the virus and include the skin, eyes, respiratory, gastrointestinal and urogenital tracts as well as the circulatory system. Some viruses localize their tissue injury in close proximity to their site of entry, particularly the viruses that infect the upper respiratory tract such as influenza, parainfluenza, rhinoviruses and coronavirus. Once the viral particle has invaded the cell, viral coded proteins direct the cell to replicate the viral genome and produce viral specific proteins. These proteins are assembled into complete virions along with the viral genome and released. In the case of enveloped viruses, the virions acquire a lipid membrane and will insert through this lipid membrane, viral specific glycoproteins. The enveloped virus families include the Herpesviridae, Retroviridae, Orthomyxoviridae, Paramyxoviridae, Flaviviridae, Togaviridae and Coronaviridae. The rhinoviruses are members of the Picornaviridae, which are not enveloped.

Viruses have evolved a number of mechanisms to enter a host cell and initiate infection. To fuse to the cell membrane, viruses have a membrane glycoprotein with membrane fusion activity. Many enveloped viruses induce a receptor-mediated endocytosis after binding to the cell surface receptor, causing the cell to form an endosomal vesicle. Once inside the vesicle, the virus particle undergoes the uncoating process. This ensures that the optimal pH for the viral genome is maintained and that the viral genome is protected from cellular nucleases.

Influenza viruses belong to the Orthomyxoviridae family of viruses and they are enveloped viruses containing negative-stranded RNA genomes with eight segments. The viral RNA encodes ten viral specific proteins. Initiation of the infective cycle requires the binding of the viral envelop to the host cell-surface receptors, followed by receptor-mediated endocytosis and the fusion of the viral and endosomal membranes. The fusion process allows the release of the viral genome into the cytoplasm, where it can migrate to the nucleus where the viral genome initiates viral transcription and replication. The protein responsible for influenza receptor binding and membrane fusion is the hemagglutinin protein (HA or H antigen). For most strains, the HA protein is the most abundant glycoprotein on the surface of the virion. The HA protein is also the target for neutralizing antibodies. There are three serotypes of influenza viruses: A, B and C. Serotypes A and B cause the majority of clinical diseases. Influenza A occurs the most frequently, it is more virulent and it is responsible for the majority of epidemics and pandemics.

Influenza A can be further subtyped based on the surface antigens HA and neuraminidase (N antigen) and the H and N antigens are the major antigenic determinants. Strains are also classified based on geographical location of the first isolate, serial number and year of isolation. Neuraminidase is an enzyme that facilitates the release of new viral particles from infected host cell. A third protein, M protein (matrix protein), is a membrane channel protein and is known as M2 in the A strains and NB in B strains. These surface viral membrane glycoproteins are the targets against which the immune system reacts.

Influenza viral particles attach to epithelial cells in the upper and lower respiratory track, where they invade the cell, release their genome and subjugate the host cell replication machinery to reproduce viral proteins and nucleic acid. Mature viral particles are released by lysis of the host cell. The resulting breaches in the respiratory epithelium results in an increase susceptibility to secondary infection. Influenza is transmitted primarily by respiratory secretions and these secretions are spread by coughing and sneezing. Influenza is also spread by direct contact when hands contaminated with the virus come in direct contact with the nasal passages or the eye. The incubation period is from 1 to 4 days and infected persons are generally infectious a day or two before symptoms appear and can remain infectious for 5 days after the onset of illness. Children and the immunocompromised shed virus for longer periods.

Influenza has been established as a serious human affliction that can cause localized epidemics and global pandemics of acute respiratory infections. Each year the influenza virus is responsible for 20,000 to 40,000 deaths and up to 300,000 hospitalization cases in the US. In the pandemic of 1918, it is widely believed that an excess of 40 million people died. Although children and younger adults experience more cases of infection, severe illness is more common in the elderly or immunocompromised individuals with chronic illnesses such as asthma, diabetes, kidney failure and heart disease. The annual epidemics run from November to March in the Northern Hemisphere and from April to September in the Southern Hemisphere.

Prophylaxis is indicated only for unvaccinated persons at high risk during an influenza outbreak. Antiviral agents have limited use due to poor tolerance and the occurrence of resistance. Presently, amantadine is the principal antiviral compound used against influenza infection, but its activity is restricted to influenza A viruses. With ongoing viral adaptation, the development of new and effective antiviral drugs against influenza A and B is of significant clinical importance. Arresting the transmission of the viruses via a nasal solution can significantly reduce rates of infections.

Prophylaxis treatments on the other hand, are used to prevent infection or lessen the severity of the disease post-exposure to the virus. Neuraminidase inhibitors lesson the symptoms of influenza infection and shorten the duration of the disease. Prophylaxis must be given within a 48-hour window of the onset of symptoms to be effective and there is a risk of resistant strains emerging.

Severe acute respiratory syndrome (SARS) was the first major new infectious disease of the 21st century. The first cases appeared in November, 2002 but it was only recognized as a new disease in March of 2003. The spread of the disease was accelerated by international air travel such that cases were reported in 22 countries. However, with modern communication technologies and a global collaborative effort the disease was contained within four months of being identified. The disease caused high morbidity and high mortality rates, with symptoms including a high fever, headache, myalgia and a dry cough. The mortality rate exceeded 60% in the over 60 age group. SARS was identified as a new Coronavirus that was responsible. The American Defense Threat Reduction Agency has also investigated silver in an unclassified report, Novel Nanotechnology-Based Antiviral Agents, written by Janice Speshock, PhD and Saber Hussain, PhD, of the Applied Biotechnology Branch 711th Human Performance Wing, Air Force Research Laboratory. They found silver interacts “inside the cell lysosomes” to inhibit the protease called cathepsin that is necessary to viral RNA replication. Bulk and nano silver have been shown “to inhibit enzyme activity” and “bind readily to thiol groups” such as cathepsin B, which “has been shown to have an essential role in Ebola and other virus replications.” Cathepsin L has an accessory role in Ebola virus replication. Drs. Speshock and Hussain note this in their August 2010 article in the Journal of Nanobiotechnology that silver “would likely make a more effective decontamination tool as opposed to an in vivo therapeutic agent.” However, embedded silver ions in nasal mucosa, accompanied by the other antimicrobial components of xylitol, quercetin, ascorbic acid, glycyrrhizic acid, turmeric, emodin, monolaurin, and grapefruit seed extract would be effective in contributing to the deactivation or reduction of viral potency.

Neeltje van Doremalen, Ph.D., et al performed research on COVID-19 and the related SARS-Cov2 virus and found that aerosol and fomite transmission of SARS-CoV-2 is plausible, since the virus can remain viable and infectious in aerosols for hours and on surfaces up to days (depending on the inoculum shed). These findings echo those with SARS-CoV-1, in which these forms of transmission were associated with nosocomial spread and super-spreading events, and they provide information for pandemic mitigation efforts.

New research has shed light on a crucial biological mechanism that may have helped the coronavirus to infect humans and spread rapidly around the world.

A detailed analysis of the virus's structure shows that the club-like “spikes” that it uses to establish infections latch on to human cells about four times more strongly than those on the related SARS coronavirus, which killed hundreds of people in a 2002 epidemic.

The finding suggests that coronavirus particles that are inhaled through the mouth have a high chance of attaching to cells in the respiratory tract, meaning that relatively few are needed for an infection to gain a foothold.

Scientists at the University of Minnesota used X-ray crystallography to create an atomic-scale 3D map of the virus's spike protein and its corresponding partner on human cells, known as the ACE-2 receptor.

When the virus encounters a human cell, the spike proteins on its surface stick to ACE-2 receptors, if the cell possess them, and allow the virus to gain access and replicate. Emodin and/or aloe emodin have been shown to compete with the ACE2 receptor to prevent covid 19 attachment.

“The 3D structure shows that compared to the virus that caused the 2002-2003 Sars outbreak, the new coronavirus has evolved new strategies to bind to its human receptor, resulting in tighter binding,” said Dr Fang Li, who led the US team. “The tight binding to the human receptor can help the virus infect human cells and spread among humans.”

Writing in the journal Nature, the researchers describe how they went on to compare the structure of the pandemic coronavirus with related strains found in bats and pangolins. They found that both animal strains could bind to the same human ACE-2 receptor, supporting previous work that suggests the human coronavirus came from bats either directly, or via pangolins that themselves became infected by bats.

Before infecting humans, the animal strains picked up key mutations that allowed the virus to spread more easily in humans. Therefore, creating an environment in the nasal mucosa which make it less receptive to binding and therefore invasion could significantly reduce infection rates.

The constant wearing of facial masks is adversely affecting dental health (periodontal disease) and the increase in dental caries. Subjects with compromised breathing (as in asthma) have shown a 500% increase in periodontal disease. The addition of bitter substances such as quinine or other bitter taste receptor stimulants has been shown to induce bronchial dilation.

Xylitol is the alcohol form of xylose, a pentose wood sugar. Since both forms are readily interchangeable, the term “xylitol/xylose” is used herein to mean “xylitol” or “xylose” or “xylitol and xylose”. Xylitol, xylose, and mixtures of xylitol and xylose are equivalent and all equally effective in equal amounts in all therapeutic uses described herein. Xylitol is present in natural chemical cycles in the body (see Touster, 1974). It has about the same safety and toxicity as table sugar (Jori, 1984). Based on measuring the amount of xylitol in the urine of a group of southern European people who are deficient in an enzyme that assists in its metabolism Touster points out that the human body uses between 5 and 15 grams of xylitol daily. Xylitol is approved by the FDA as a food additive and is widely used as a sweetener especially in chewing gums.

In 1998 Kontiokari found that a 2.5 percent solution of xylitol/xylose decreased the adherence of this bacteria when present either in the nasal mucosal cell or in the bacteria. When a five percent solution was present in both the bacteria and the mucosal cell, adherence of strep pneumonia, the major pathogen, was reduced by two-thirds; from an average of 41 bacteria per cell to 13 (Kontiokari, 1998). His article concludes by stating: “These observations are consistent with the fact that monosaccharides are able to inhibit adherence only at the high concentrations, that are easily achieved in the oral cavity. The worldwide spread of penicillin-resistant strains of pneumococci substantiates the need for new approaches to preventing bacterial infections. Xylitol seems to be a promising agent for this purpose.”

Matti Uhari, one of Kontiokari's colleagues in Finland has been studying the effects of oral xylitol/xylose in reducing the incidence of recurrent otitis as disclosed in U.S. Pat. No. 5,719,196 (Uhari, 1996; Uhari, 1998). Uhari's original study looked at the effect of xylitol chewing gum in reducing the incidence of otitis. The highest incidence of otitis is in infants less than two who cannot chew gum. Uhari subsequently studied the incidence of otitis in children getting an oral solution of xylitol. He found between a thirty and forty-percent reduction in the incidence of otitis using these supplements.

A nasal spray may be formulated having approximately 10% xylitol/xylose in an aqueous solution. The spray is administered by a conventional spray bottle. See, U.S. Pat. No. 6,054,143. As little as 1% xylitol/xylose in solution appears to be the effective minimum strength, the maximum strength is a saturated solution of 64 grams of xylitol/xylose per 100 ml of solution.

The solution may be slightly hypertonic (0.45% to 0.95% sodium chloride). Mixing in a saline aqueous solution to facilitate the washing effect of the saline, the saline solution should be slightly hypotonic.

When administered, the objective is to reduce one or more of the severity of the viral illness, the severity of symptoms of the viral illness, and the incidence of symptoms of the viral illness relative to a mammal to which the composition has not been administered.

### SUMMARY OF THE INVENTION

The present invention relates the prophylactic use of a composition to reduce the incidence of contraction of illnesses caused by microbial organisms. More particularly, the present invention relates to methods for treating, reducing or preventing one or more symptoms or adverse effects of a microbial infection and to methods for reducing the infectivity or transmission of microbial infections.

The composition comprises silver, preferably in a colloidal or nanoparticle form, and further comprises glycyrrhizic acid and quercetin.

A base vehicle of xylitol may be employed. The silver (e.g., colloidal or nanoparticle) may be complexed with a protein, glycyrrhizic acid and quercetin. Additional ingredients may include ascorbic acid (vitamin C), curcumin, grapefruit seed extract, monolaurin, eucalyptol and emodin/aloe emodin, and quinine with flavoring and acceptable preservative system.

The formulation may be administered topically, as a nasal spray, mouthwash, sublingually, or bronchial aerosol.

The formulation may also be applied to surfaces and inanimate objects, such as respirators and masks.

Accordingly, it is an object of certain embodiments of the invention to provide a method for reducing the incidence of contracting an illness caused by a microbial organism.

In the first aspect, the present invention relates to a method for the prophylactic use of an anti-microbial composition to reduce the incidence of contracting an illness via an or spray application. The method comprises the steps of administering to a mammal that has been, or will be, exposed to an illness caused by a virus or microbe, an amount of an anti-viral or anti-microbial composition having a first ingredient referred to as xylitol; and a second ingredient colloidal/nano particle silver complexed with protein, glycyrrhizic acid and quercetin.

Additional ingredients include vitamin C, curcumin, grapefruit seed extract, monolaurin, eucalyptol and emodin/aloe emodin, and quinine with flavoring and acceptable preservative system.

The anti-microbial composition is effective, when administered as an oral spray and/or as a throat spray to a mammal that will be exposed to an illness caused by a microbe, to reduce the incidence of contracting said illness.

In a first aspect, the present invention relates to a composition. The composition of the present invention includes the ingredients purified water, xylitol, silver, glycyrrhizic acid, ascorbic acid, quercetin, grapefruit seed extract, curcumin, zinc, eucalyptol, quinine or similar bitter herb to stimulate bitter taste receptors, and emodin/aloe emodin in a pH adjusted and preserved an aqueous base.

As used herein, the term “acceptable” means a component that is suitable for use with humans and/or animals without undue adverse side effects (such as toxicity, irritation, and allergic responses), commensurate with a reasonable risk/benefit ratio.

The term “safe and effective amount” refers to the quantity of a component, which is sufficient to yield a desired therapeutic response without undue adverse side effects (such as toxicity, irritation, or allergic responses), commensurate with a reasonable risk/benefit ratio when used in the manner described herein.

The term “inhibiting” a microbe, as used herein, refers to reducing or preventing further growth of the microbe, or preventing the microbe from attaching to normal cells, and/or the elimination of some or all of the infectious particles from the human or animal being treated. Suitable methods for determining microbe inhibition are discussed in the examples.

The term “transmissivity” as used herein refers to the transfer of a microbe from one host to another.

In a first embodiment, the composition of the present invention includes a first ingredient xylitol, and a second ingredient silver complex from a combination of colloidal and nano-processed silver technology, ascorbic acid, quercetin, grapefruit seed extract zinc, quinine, and curcumin, in a safe and effective amount to provide one or more of the beneficial effects described herein. The ingredients glycyrrhizic acid, eucalyptol, and monolaurin inhibit NF-κB activation, which plays an important role in viral replication. Emodin prevents binding of corona viruses to the ACE2 receptor.

The processes for the preparation of pharmacologically or biologically active plant extracts in a convenient, administrable dosage form from any of the plants mentioned above, are well known in the art.

The composition of the present invention may be used to treat viral infection, since the composition of the present invention has significant antimicrobial properties as demonstrated by the examples of this application. The composition of the present invention may also be used as a therapeutic composition to treat one or more symptoms of a viral infection, including sore throat, congestion, laryngitis, mucositis, and/or mucous membrane inflammation by administration to a patient suffering from one or more of these symptoms or ailments.

The composition of the present invention may be used to treat bacterial infection secondary to viral infection, or concurrent with the viral infection.

The composition of the present invention may also be employed to reduce the incidence of contracting an illness. In this application of the composition of the present invention, a safe and effective amount of the composition of the present invention is administered to a mammal that has been or will be exposed to an illness caused by a microbe, to reduce the incidence of contracting said illness, relative to a mammal that has been or will be exposed to an illness caused by a microbe to which the composition of the present invention has not been administered.

The composition of the present invention may be formulated in other liquid forms such as syrups, mouthwashes or sprays, topical sprays for porous or hard surfaces, with variations in the solvent or dispersant depending on application.

Preferably, the treatment time is about 5 to 10 minutes, so as to permit a prolonged contact of the composition with oral and throat tissues. Alternatively, such formulations can be in a concentrated form suitable for dilution with water or other materials prior to use as a spray or dispersed via a humidifier.

The composition of the present invention may also be formulated into an inhalant composition. Such a composition may be prepared using well-known techniques. For these types of formulations, suitable carriers may include the following ingredients: saline with one or more preservatives, absorption promoters to enhance bioavailability, fluorocarbons, and/or conventional solubilizing or dispersion agents.

The yellow pigment of the rhizome of turmeric is composed of three compounds known as curcuminoids. The three curcuminoids are curcumin (diferuloylmethane), desmethoxycurcumin (hydroxycinnamoyl feruloylmethane), and bis-desmethoxycurcumin (dihydroxydicinnamoyl methane) The ingredient of the composition of the present invention, obtained from turmeric, preferably includes curcuminoids, such as curcumin (diferuloylmethane), desmethoxycurcumin (hydroxycinnamoyl feruloylmethane), and bis-desmethoxycurcumin (dihydroxydicinnamoyl methane), and mixtures of two or more of these curcuminoids.

Reducing or preventing transmission relates to preventing or reducing the spread of a microbe from one patient (infected) to another patient (non-infected). Some patients may be considered carriers of the infection. Carriers are individuals who actively shed microbes but do not suffer from an acute infection. These carriers may be said to be persistently (or chronically) infected with the microbe. In addition to the persistently infected shedder, other infective individuals may be those who are actively infected, and particularly those in the early or late stages of an acute infection. One aspect of the invention relates to administering to a mammal prior to infection with a microbe, a composition of the present invention, to prevent the spread of the disease to other mammals and/or potentially reduce the symptoms of the disease in the infected mammal.

Prophylactic treatment is aimed at a patient that will soon be exposed to a microbe or has recently been exposed to a microbe. Such prophylactic treatment may be effective either alone, or to augment a vaccine. Prophylactic treatment may also be used against microbes for which there is not yet a vaccine available. In the case of prophylactic treatment, the composition of the invention is administered to a patient that will be exposed to a microbe or has recently been exposed to a microbe for the purpose of reducing the incidence of active infection by the microbe in that patient.

In another aspect, the present invention relates to a method of reducing, treating or preventing of at least one symptom or adverse effect of viral infection by administering, to a patient infected with a virus, a composition of the present invention.

In the method, the patient may be a human, an in vitro cell system, or an animal. Preferably, the patient is a mammal, more preferably, a human. In the method, the virus that may be inhibited by administration of the composition of the present invention includes, among other viruses, rhinoviruses, influenza viruses, adenovirus, coronavirus, influenza virus, rubella virus, and respiratory syncytial virus (RSV). In a preferred embodiment, the viruses that may be inhibited by administration of the composition include at least human rhinovirus 16, corona COVID-19 virus, MERS, SARS, the common cold virus corona 229E, and Influenza A.

The symptoms, caused by a viral infection, that may be treated, reduced, or at least partially prevented by this method of the present invention, may include one or more of headache, joint pain, fever, cough, sneezing, muscle ache, running nose, dry mouth, dizziness, and other symptoms related to viral infection.

The effective amount of the composition will vary depending on such factors as the patient being treated, the particular mode of administration, the activity of the particular active ingredients employed, the age, general health, sex and diet of the patient, time of administration, rate of excretion, the particular combination of ingredients employed, the total content of the main ingredient of the composition, and the severity of the illness or symptom. It is within the skill of the person of ordinary skill in the art to account for these factors.

The composition may be administered about 1 to about 10 times per day, as needed, or most preferably, about 3 to about 10 times per day, as needed, when the human is exposed in public or infection-prone areas such as a hospital or medical clinic. In environments where health care professionals are exposed in an airborne viral-rich enclosed area, it is recommended that the spray be used every 30 minutes along with other preventive methods such as hand washing, frequent changes of N95 masks and protective clothing (PPE), cleaning of eyewear, and room filtration/ventilation.

When the composition is administered as a spray, the amounts each of the active ingredients may be reduced as the spray composition delivers the active ingredients more directly to the location where they are needed.

Glycyrrhizic acid (GA), belonging to a class of triterpenes, is a conjugate of two molecules, namely glucuronic acid and glycyrrhetinic acid. It is naturally extracted from the roots of licorice plants. With its more common uses in the confectionery and cosmetics industry, GA extends its applications as an herbal medicine for a wide range of ailments. At low appropriate doses, anti-inflammatory, anti-diabetic, antioxidant, anti-tumor, antimicrobial and anti-viral properties have been reported by researchers worldwide.

CN103705969 describes a method for preparing a chitosan-based silver-loaded composite antimicrobial superfine fiber membrane comprising polyoxyethylene or polyvinyl alcohol.

RU2545735 (C1) discloses a hydrogel-based wound dressing, which contains antimicrobial and antioxidant ingredients: such as montmorillonite modified with silver and fulerenol, in order to improve the process of regeneration and prevent/reduce infections.

CN103446618 (A) comprises a hydrogel with antibacterial activity and its method of preparation. The hydrogel consists of 0.1-5% of silver norfloxacin, 5-30% of polymer, 0-10% of plasticizer and water. This hydrogel enables absorption of exudates and the prevention of infections.

CN101278896 (B) describes the production of a chitosan gel containing nanoparticles of silver used for treating the inflammatory and infectious processes associated with skin lesions.

US 20200163990 relates to metal, e.g., silver antibiotic compositions. See also, US 20200157266, 20200154712; 20200138851; 20200095421.

The following preferred ranges define compositions according to the invention that are suited for administration in a spray formulation according to the methods of the invention.

These and various other advantages and features of novelty that characterize the invention are pointed out with particularity in the claims annexed hereto and forming a part hereof. However, for a better understanding of the invention, its advantages, and the objects obtained by its use, reference should be made to the accompanying descriptive matter, in which there is described a preferred embodiment of the invention.

In some embodiments, the nasal spray pharmaceutical formulation comprises one or more absorption enhancement agents; and optionally one or more agents selected from isotonicity agents; stabilizing agents; preservatives; taste-masking agents; viscosity modifiers; antioxidants; buffers and pH adjustment agents; wherein the pH of the nasal spray pharmaceutical formulation is between about 2.0 and about 6.0.

In some embodiments, the nasal spray pharmaceutical formulation has a pH between about 3.0 and about 5.0. In some embodiments, the nasal spray pharmaceutical formulation has a pH of about 4.0. In some embodiments, the nasal spray pharmaceutical formulation comprises pH adjustment agents. In some embodiments, the pH adjustment agent is an acid, a base, a buffer, or a combination thereof. In some embodiments, the acid is adipic acid, ammonium chloride, citric acid, acetic acid, hydrochloric acid, lactic acid, phosphoric acid, propionic acid, sulfuric acid or tartaric acid; the base is sodium hydroxide, sodium citrate, sodium bicarbonate, sodium carbonate; and the buffer is a phosphate buffer, acetate buffer, or citrate buffer. In some embodiments, the acid is hydrochloric acid.

In some embodiments, the nasal spray formulation comprises one or more absorption enhancers selected from dodecyl maltoside, benzalkonium chloride, oleic acid, or salt thereof, polysorbate 20, polysorbate 80, and sodium lauryl sulfate.

In some embodiments, the formulation comprises one or more absorption enhancers selected from alcohol, aprotinin, benzalkonium chloride, benzyl alcohol, capric acid, ceramides, cetylpyridinium chloride, chitosan, cyclodextrins, deoxycholic acid, decanoyl, dimethyl sulfoxide, glyceryl monooleate, glycofurol, glycofurol, glycosylated sphingosines, glycyrrhetinic acids, 2-hydroxypropyl-β-cyclodextrin, laureth-9, lauric acid, lauroyl carnitine, lysophosphatidylcholine, menthol, poloxamer 407 or F68, poly-L-arginine, polyoxyethylene-9-lauryl ether, isopropyl myristate, isopropyl palmitate, lanolin, light mineral oil, linoleic acid, menthol, myristic acid, myristyl alcohol, oleic acid, or salt thereof, oleyl alcohol, palmitic acid, polysorbate 20, polysorbate 80, propylene glycol, polyoxyethylene alkyl ethers, polyoxylglycerides, pyrrolidone, quillaia saponin, salicylic acid, sodium salt, β-sitosterol 3-D-glucoside, sodium lauryl sulfate, sucrose cocoate, taurocholic acid, taurodeoxycholic acid, taurodihydrofusidic acid, thymol, tricaprylin, triolein, and alkylsaccharides.

In some embodiments, the formulation comprises one or more absorption enhancers selected from dodecyl maltoside, benzalkonium chloride, oleic acid, or salt thereof, polysorbate 20, polysorbate 80, and sodium lauryl sulfate.

In some embodiments, the nasal spray pharmaceutical formulation comprises an isotonicity agent. In some embodiments, the isotonicity agent is dextrose, glycerin, mannitol, potassium chloride, or sodium chloride. In some embodiments, the isotonicity agent is sodium chloride.

In some embodiments, the nasal spray formulation additionally comprises a stabilizing agent. In some embodiments, the stabilizing agent is ethylenediaminetetraacetic acid (EDTA) or a salt thereof. In some embodiments, the EDTA is disodium EDTA. In some embodiments, the nasal spray formulation comprises from about 0.001% (w/v) to about 1% (w/v) of disodium EDTA.

In some embodiments, the nasal spray formulation comprises one or more absorption enhancers selected from alkylglycosides, benzalkonium chloride, oleic acid, or salt thereof, polysorbate 20, polysorbate 80, sodium lauryl sulfate, cyclodextrins, medium and long chain fatty acids, or salts thereof, saturated and unsaturated fatty acids, or salts thereof, alcohol, glycerin, propylene glycol, PEG 300/400, and benzyl alcohol.

In some embodiments, the nasal spray formulation further comprises an antioxidant. In some embodiments, the nasal spray formulation further comprises an antioxidant selected from alpha tocopherol, arachidonic acid, ascorbic acid, ascorbyl palmitate, benzethonium chloride, benzethonium bromide, benzalkonium chloride, butylated hydroxyanisole (BHA), butylated hydroxytoluene (BHT), capric acid, caproic acid, carbon dioxide, cetylpyridium chloride, chelating agents, chitosan derivatives, citric acid monohydrate, dodecyl dimethyl aminopropionate, enanthic acid, erythorbic acid, ethyl oleate, fumaric acid, glycerol oleate, glyceryl monostearate, lauric acid, limonene, linolenic acid, lysine, malic acid, menthol, methionine, monothioglycerol, myristic acid, oleic acid, palmitic acid, pelargonic acid, peppermint oil, phosphoric acid, polysorbates, potassium metabisulfite, propionic acid, propyl gallate, sodium ascorbate, sodium bisulfite, sodium caprate, sodium desoxycholate, sodium deoxyglycolate, sodium formaldehyde sulfoxylate, sodium glycocholate, sodium hydroxybenzoyal amino caprylate, sodium lauryl sulfate, sodium metabisulfite, sodium sulfite, sodium taurocholate, sodium thiosulfate, stearic acid, sulfur dioxide and a combination thereof.

In some embodiments, the nasal spray formulation further comprises synergists with the antioxidants selected from citric acid monohydrate, tartaric acid, thymol, tocopherol (alpha tocopherol), tocopherasol, vitamin E and vitamin E polyethylene glycol succinate and a combination thereof.

In some embodiments, the nasal spray formulation further comprises permeation enhancers selected from alcohol, arachidonic acid, benzethonium chloride, benzethonium bromide, benzalkonium chloride, capric acid, caproic acid, carvone, cetylpyridium chloride, chitosans, citric acid, 6-cyclohexyl-1-hexyl-β-D-maltopyranoside, n-decyl-β-D-maltopyranoside, dimethyl sulfoxide, dodecyl dimethyl aminopropionate, 1-O-n-Dodecyl-β-D-maltopyranoside, dodecylpolyethyleneglycolether, edetate disodium dihydrate, enanthic acid, glycerylmonooleate, glyceryl monostearate, glycofurol, isopropyl myristate, isopropyl palmitate, pelargonic acid, lanolin, lauric acid, light mineral oil, limonene, linoleic acid, lysine, menthol, myristic acid, myristyl alcohol, oleic acid, oleyl alcohol, palmitic acid, peppermint oil, polyoxyethylene alkyl ethers, polyoxylglycerides, polysorbates, pyrrolidone, sodium caprate, sodium desoxycholate, sodium deoxyglycolate, sodium glycocholate, sodium hydroxybenzoyal amino caprylate, sodium lauryl sulfate, sodium taurocholate, stearic acid, thymol, tricaprylin, triolein, undecylenic acid, and a combination thereof.

In some embodiments, the nasal spray formulation comprises: about 0.001% to 1% of any one of the antioxidants described herein, or a combination of any one of the antioxidants described herein.

In some embodiments, the nasal spray formulation comprises a buffering agent.

Buffering agents include, but are not limited to, adipic acid, boric acid, calcium carbonate, calcium hydroxide, calcium lactate, calcium phosphate, tribasic, citric acid monohydrate, dibasic sodium phosphate, diethanolamine, glycine, maleic acid, malic acid, methionine, monobasic sodium phosphate, monoethanolamine, monosodium glutamate, phosphoric acid, potassium citrate, sodium acetate, sodium bicarbonate, sodium borate, sodium carbonate, sodium citrate dihydrate, sodium hydroxide, sodium lactate, and triethanolamine.

In some embodiments, the nasal spray formulation is an aqueous solution, aqueous suspensions, aqueous emulsion, non-aqueous solution, non-aqueous suspension or non-aqueous emulsion.

In some embodiments, the nasal spray pharmaceutical formulation comprises one or more absorption enhancement agents; and optionally one or more agents selected from isotonicity agents; stabilizing agents; preservatives; taste-masking agents; viscosity modifiers; antioxidants; buffers and pH adjustment agents; wherein the pH of the nasal spray pharmaceutical formulation is between about 2.0 and about 6.0. In some embodiments, the nasal spray pharmaceutical formulation has a pH between about 3.0 and about 5.0. In some embodiments, the nasal spray pharmaceutical formulation has a pH of about 4.0.

In some embodiments, the nasal spray pharmaceutical formulation comprises pH adjustment agents. In some embodiments, the pH adjustment agent is an acid, a base, a buffer, or a combination thereof. In some embodiments, the acid is adipic acid, ammonium chloride, citric acid, acetic acid, hydrochloric acid, lactic acid, phosphoric acid, propionic acid, sulfuric acid or tartaric acid; the base is sodium hydroxide, sodium citrate, sodium bicarbonate, sodium carbonate; and the buffer is a phosphate buffer, acetate buffer, or citrate buffer.

In some embodiments, the composition further comprises a membrane penetration-enhancing agent. In some embodiments, the membrane penetration-enhancing agent is a surfactant, a bile salt, a phospholipid, an alcohol, an enamine, a long-chain amphipathic molecule, a small hydrophobic molecule, sodium or a salicylic acid derivative, a glycerol ester of acetoacetic acid, a cyclodextrin, a medium-chain or long chain fatty acids, a chelating agent, an amino acid or salt thereof, an enzyme or combination thereof. In some embodiments, the membrane penetration-enhancing agent is selected from the group consisting of citric acid, sodium citrate, propylene glycol, glycerin, ascorbic acid, sodium metabisulfite, ethylenediaminetetraacetic acid (EDTA) disodium, benzalkonium chloride, hydroxyquinolone, sodium hydroxide, and combinations thereof. In some embodiments, the membrane penetration-enhancing agent is selected from the group consisting of citric acid, sodium citrate, propylene glycol, glycerin, ascorbic acid, sodium metabisulfite, ethylenediaminetetraacetic acid (EDTA) disodium, benzalkonium chloride, sodium hydroxide, and combinations thereof. In some embodiments, membrane penetration-enhancing agent is benzalkonium chloride, EDTA, or a combination thereof.

Exemplary mucosal delivery enhancing agents include the following agents and any combinations thereof: (a) an aggregation inhibitory agent; (b) a charge-modifying agent; (c) a pH control agent; (d) a degradative enzyme inhibitory agent; (e) a mucolytic or mucus clearing agent; (f) a ciliostatic agent; (g) a membrane penetration-enhancing agent selected from: (i) a surfactant; (ii) a bile salt; (ii) a phospholipid additive, mixed micelle, liposome, or carrier; (iii) an alcohol; (iv) an enamine; (v) an NO donor compound; (vi) a long-chain amphipathic molecule; (vii) a small hydrophobic penetration enhancer; (viii) sodium or a salicylic acid derivative; (ix) a glycerol ester of acetoacetic acid; (x) a cyclodextrin or beta-cyclodextrin derivative; (xi) a medium-chain fatty acid; (xii) a chelating agent; (xiii) an amino acid or salt thereof; (xiv) an N-acetylamino acid or salt thereof; (xv) an enzyme degradative to a selected membrane component; (ix) an inhibitor of fatty acid synthesis; (x) an inhibitor of cholesterol synthesis; and (xi) any combination of the membrane penetration enhancing agents recited in (i)-(x); (h) a modulatory agent of epithelial junction physiology; (i) a vasodilator agent; (j) a selective transport-enhancing agent; and (k) a stabilizing delivery vehicle, carrier, mucoadhesive, support or complex-forming species with which the compound is effectively combined, associated, contained, encapsulated or bound resulting in stabilization of the compound for enhanced nasal mucosal delivery, wherein the formulation of the compound with the intranasal delivery-enhancing agents provides for increased bioavailability of the compound in a blood plasma of a subject.

Additional mucosal delivery-enhancing agents include, for example, citric acid, sodium citrate, propylene glycol, glycerin, ascorbic acid (e.g., L-ascorbic acid), sodium metabisulfite, ethylenediaminetetraacetic acid (EDTA) disodium, benzalkonium chloride, sodium hydroxide, and mixtures thereof. For example, EDTA or its salts (e.g., sodium or potassium) are employed in amounts ranging from about 0.01% to 2% by weight of the composition containing alkylsaccharide preservative.

Liquid preparations include solutions, suspensions and emulsions, for example, water, or water-ethanol, or water-propylene glycol solutions. Typically, the formulation is an aqueous liquid solution. Additional ingredients in liquid preparations may include preservatives, stabilizing agents, tonicity agents, absorption enhancers, pH-adjusting agents, antioxidants, buffers, sweeteners/flavoring agents/task-masking agents, and optionally other ingredients. Ingredients in liquid preparations may serve different functions. The function(s) of a particular ingredient will depend on a number of factors including, but not limited to, presence or absence of other ingredients, concentration(s), and other factors.

Preservatives include: benzalkonium chloride, methylparaben, sodium benzoate, benzoic acid, phenyl ethyl alcohol, and the like, and mixtures thereof. Due to their chemical properties, certain preservatives can function as a surfactants and/or absorption enhancers in certain circumstances, depending on concentration in the formulation and other factors.

Other preservatives include: alcohol, benzalkonium chloride, benzethonium chloride, benzoic acid, benzyl alcohol, boric acid, bronopol, butylated hydroxyanisole (BHA), butylene glycol, butylparaben, calcium acetate, calcium chloride, calcium lactate, carbon dioxide, bentonite, cetrimide, cetylpyridinium chloride, chlorhexidine, chlorobutanol, chlorocresol, chloroxylenol, citric acid monohydrate, cresol, dimethyl ether, ethylparaben, glycerin, hexetidine, imidurea, magnesium trisilicate, isopropyl alcohol, lactic acid, methylparaben, monothioglycerol, parabens (methyl, ethyl and propyl), pentetic acid, phenol, phenoxyethanol, phenylethyl alcohol, phenylmercuric acetate, phenylmercuric borate, phenylmercuric nitrate, potassium benzoate, potassium metabisulfite, potassium sorbate, propionic acid, propylgallate, propylene glycol, propylparaben, propylparaben sodium, sodium acetate, sodium benzoate, sodium borate, sodium lactate, sodium metabisulfite, sodium propionate, sodium sulfite, sorbic acid, sulfobutyletherb-cyclodextrin, sulfur dioxide, edetic acid, thimerosal, and xylitol.

In some embodiments, preservatives include, but are not limited to, antibacterial agents, antifungal agents, and antioxidants.

Antibacterial agents include, but are not limited to, chlorocresol, diazolidinyl urea, dimethyl sulfoxide, glacial acetic acid, imidurea, iodine/edetic acid, phenylmercuric acetate, phenylmercuric borate, phenylmercuric hydroxide, potassium sorbate, sodium hydroxide, sorbic acid, thymol, antiseptics, and disinfectants.

Antifungal agents include, but are not limited to, benzoic acid, butylene glycol, butylparaben, chlorocresol, coconut oil, dimethyl sulfoxide, ethylparaben, glacial acetic acid, imidurea, methylparabens, phenylmercuric acetate, phenylmercuric borate, phenylmercuric hydroxide, potassium sorbate, propylparaben, sodium propionate, sodium thiosulfate, thymol, and vanillin.

Surfactants include but are not limited to: Polysorbate 80 NF, polyoxyethylene 20 sorbitan monolaurate, polyoxyethylene (4) sorbitan monolaurate, polyoxyethylene 20 sorbitan monopalmitate, polyoxyethylene 20 sorbitan monostearate, polyoxyethylene (4) sorbitan monostearate, polyoxyethylene 20 sorbitan tristearate, polyoxyethylene (5) sorbitan monooleate, polyoxyethylene 20 sorbitan trioleate, polyoxyethylene 20 sorbitan monoisostearate, sorbitan monooleate, sorbitan monolaurate, sorbitan monopalmitate, sorbitan monostearate, sorbitan trilaurate, sorbitan trioleate, sorbitan tristearate, and the like, and mixtures thereof. Due to their chemical properties, certain surfactants can function as a preservatives and/or absorption enhancers in certain circumstances, depending on concentration in the formulation and other factors.

Surfactants include but are not limited to: cationic, anionic, nonionic and zwitterionic surfactants. Surfactants also include: anionic surfactants (e.g. carboxylates sulphonates, petroleum sulphonates, alkylbenzenesulphonates, naphthalenesulphonates, olefin sulphonates, alkyl sulphates, sulphates, sulphated natural oils and fats, sulphated esters, sulphated alkanolamides, alkylphenols, ethoxylated and sulphated), nonionic surfactants (e.g. ethoxylated aliphatic alcohol, polyoxyethylene surfactants, carboxylic esters, polyethylene glycol esters, anhydrosorbitol ester and it's ethoxylated derivatives, glycol esters of fatty acids, carboxylic amides, monoalkanolamine condensates, polyoxyethylene fatty acid amides), cationic surfactants (e.g. quaternary ammonium salts, amines with amide linkages, polyoxyethylene alkyl and alicyclic amines, 4.n,n,n′,n′ tetrakis substituted ethylenediamines, 2-alkyl 1-hydroxethyl2-imidazolines), amphoteric surfactants (amphoteric surfactants contains both an acidic and a basic hydrophilic moiety in their surface e.g., n-coco 3-aminopropionic acid/sodium salt, n-tallow 3-iminodipropionate, disodium salt, n-carboxymethyl n dimethyl n-9 octadecenyl ammonium hydroxide, n-cocoamidethyl n hydroxyethylglycine, sodium salt, etc.).

Antioxidants include, but are not limited to, tocopherol, arachidonic acid, ascorbic acid, ascorbyl palmitate, benzethonium chloride, benzethonium bromide, benzalkonium chloride, butylated hydroxyanisole (BHA), butylated hydroxytoluene (BHT), capric acid, caproic acid, carbon dioxide, cetylpyridium chloride, chelating agents, chitosan derivatives, citric acid monohydrate, dodecyl dimethyl aminopropionate, enanthic acid, erythorbic acid, ethyl oleate, fumaric acid, glycerol oleate, glyceryl monostearate, lauric acid, limonene, linolenic acid, lysine, malic acid, menthol, methionine, monothioglycerol, myristic acid, oleic acid, or salt thereof, palmitic acid, pelargonic acid, peppermint oil, phosphoric acid, polysorbates, potassium metabisulfite, propionic acid, propyl gallate, sodium ascorbate, sodium bisulfite, sodiumcaprate, sodium desoxycholate, sodium deoxyglycolate, sodium formaldehyde sulfoxylate, sodium glycocholate, sodium hydroxybenzoyal amino caprylate, sodium lauryl sulfate, sodium metabisulfite, sodium sulfite, sodium taurocholate, sodium thiosulfate, stearic acid, sulfur dioxide and a combination thereof.

Buffers include, but are not limited to, phosphate buffers, acetate buffers, and citrate buffers.

In some embodiments, the nasal spray formulation comprises a buffering agent. Buffering agents include, but are not limited to, adipic acid, boric acid, calcium carbonate, calcium hydroxide, calcium lactate, calcium phosphate, tribasic, citric acid monohydrate, dibasic sodium phosphate, diethanolamine, glycine, maleic acid, malic acid, methionine, monobasic sodium phosphate, monoethanolamine, monosodium glutamate, phosphoric acid, potassium citrate, sodium acetate, sodium bicarbonate, sodium borate, sodium carbonate, sodium citrate dihydrate, sodium hydroxide, sodium lactate, and triethanolamine.

Isotonicity agents include sodium chloride, calcium chloride, magnesium chloride, sorbitol, sucrose, dextrose, lactose, mannitol, trehalose, raffinose, polyethylene glycol, hydroxyethyl starch, glycerine, glycine, and the like, and mixtures thereof. In certain embodiments, the isotonicity agent is chosen from dextrose, glycerin, mannitol, potassium chloride, and sodium chloride. In certain embodiments, the isotonicity agent is sodium chloride.

In certain embodiments, the formulations disclosed herein contain sodium chloride in an amount sufficient to cause the final composition to have a nasally acceptable osmolality, preferably 240-350 mOsm/kg. In certain embodiments, the formulations contain 0.3-1.9% sodium chloride.

Sweetners/flavoring agents/task-masking agents include, but are not limited to, sucrose, dextrose, lactose, sucralose, acesulfame-K, aspartame, saccharin, sodium saccharin, citric acid, aspartic acid, eucalyptol, mannitol, glycerin, xylitol, menthol, glycyrrhizic acid, cinnamon oils, oil of wintergreen, peppermint oils, clover oil, bay oil, anise oil, eucalyptus, vanilla, citrus oil such as lemon oil, orange oil, grape and grapefruit oil, fruit essences including apple, peach, pear, strawberry, raspberry, cherry, plum, pineapple, apricot, etc. and combinations thereof. In some embodiments, the formulations contain from about 0.0001 percent to about 1 percent of a sweetener/flavoring agent/task-masking agent, and may be present at lower or higher amounts as a factor of one or more of potency of the effect on flavor, solubility of the flavorant, effects of the flavorant on solubility or other physicochemical or pharmacokinetic properties of other formulation components, or other factors.

In certain embodiments, the pharmaceutical formulation additionally comprises an isotonicity agent. The intranasal formulation may comprise between about 0.2% (w/v) and about 1.2% (w/v) isotonicity agent, such as about 0.2% (w/v), about 0.3% (w/v), about 0.4% (w/v), about 0.5% (w/v), about 0.6% (w/v), about 0.7% (w/v), about 0.8% (w/v), about 0.9% (w/v), about 1.0% (w/v), about 1.1% (w/v), or about 1.2% (w/v). The intranasal formulation may comprise more than about 0.1% (w/v) isotonicity agent. The intranasal formulation may comprise less than about 1.2% (w/v) isotonicity agent. In other embodiments, the intranasal formulation may comprise between about 0.2% (w/v) and about 1.9% (w/v) isotonicity agent, such as about 0.2% (w/v), about 0.3% (w/v), about 0.4% (w/v), about 0.5% (w/v), about 0.6% (w/v), about 0.7% (w/v), about 0.8% (w/v), about 0.9% (w/v), about 1.0% (w/v), about 1.1% (w/v), about 1.2% (w/v), about 1.3% (w/v), about 1.4% (w/v), about 1.5% (w/v), about 1.6% (w/v), about 1.7% (w/v), about 1.8% (w/v), or about 1.9% (w/v). The intranasal formulation may comprise less than about 1.9% (w/v) isotonicity agent.

In certain embodiments, the formulation additionally comprises an absorption enhancer. In certain embodiments, the pharmaceutical formulation comprises between about 0.005% (w/v) to about 2.5% (w/v) of the absorption enhancer. In certain embodiments, the pharmaceutical formulation comprises between about 0.05% (w/v) to about 2.5% (w/v) of the absorption enhancer. In certain embodiments, the pharmaceutical formulation comprises between about 0.1% (w/v) to about 0.5% (w/v) of the absorption enhancer. In certain embodiments, the pharmaceutical formulation comprises about 0.25% (w/v) of the absorption enhancer. In certain embodiments, the pharmaceutical formulation comprises about 0.18% (w/v) of the absorption enhancer.

In certain embodiments, the absorption enhancer is selected from benzalkonium chloride, cyclodextrins, chitosan, deoxycholic acid, an alkylsaccharide (e.g., a nonionic alkylsaccharide surfactant such as an alkylglycoside and a sucrose ester of fatty acids that consists of an aliphatic hydrocarbon chain coupled to a sugar moiety by a glycosidic or ester bond, respectively), fusidic acid derivatives, glycocholic acid, laureth-9, phosphatidylcholines, taurocholic acid, taurodihydrofusidic acid, microspheres and liposomes, and bile salts. In certain embodiments, the absorption enhancer is benzalkonium chloride. The formulation may comprise about 0.01% (w/v) to about 1% (w/v) benzalkonium chloride. In certain embodiments, the pharmaceutical formulation comprises about 0.005% (w/v) to about 0.015% (w/v) benzalkonium chloride. In certain embodiments, the pharmaceutical formulation comprises about 0.01% (w/v), about 0.02% (w/v), about 0.03% (w/v), or about 0.04% (w/v) of benzalkonium chloride. In certain embodiments, the pharmaceutical formulation comprises about 0.01% (w/v) benzalkonium chloride. In certain embodiments, the pharmaceutical formulation comprises about 0.02% (w/v) benzalkonium chloride. In certain embodiments, the pharmaceutical formulation comprises about 0.04% benzalkonium chloride.

In certain embodiments, the pharmaceutical formulation comprises benzalkonium chloride in an amount between about 0.001% (w/v) and about 1% (w/v). In certain other embodiments, the pharmaceutical formulation comprises benzalkonium chloride in an amount between about 0.001% (w/v) and about 0.5% (w/v). In certain other embodiments, the pharmaceutical formulation comprises benzalkonium chloride in an amount between about 0.001% (w/v) and about 0.2% (w/v). In some embodiments, the pharmaceutical formulation comprises 0.001% (w/v), 0.003% (w/v), 0.005% (w/v), 0.007% (w/v), 0.009% (w/v), 0.01% (w/v), 0.02% (w/v), 0.03% (w/v), 0.04% (w/v), 0.05% (w/v), 0.06% (w/v), 0.07% (w/v), 0.08% (w/v), 0.09% (w/v), 0.1% (w/v), 0.11% (w/v), 0.12% (w/v), 0.13% (w/v), 0.14% (w/v), 0.15% (w/v), 0.16% (w/v), 0.17% (w/v), 0.18% (w/v), 0.19% (w/v), 0.2% (w/v), 0.31% (w/v), 0.22% (w/v), 0.23% (w/v), 0.24% (w/v), 0.25% (w/v), 0.26% (w/v), 0.27% (w/v), 0.28% (w/v), 0.29% (w/v), 0.3% (w/v), 0.31% (w/v), 0.32% (w/v), 0.33% (w/v), 0.34% (w/v), 0.35% (w/v), 0.36% (w/v), 0.37% (w/v), 0.38% (w/v), 0.39% (w/v), 0.4% (w/v), 0.41% (w/v), 0.42% (w/v), 0.43% (w/v), 0.44% (w/v), 0.45% (w/v), 0.46% (w/v), 0.47% (w/v), 0.48% (w/v), 0.49% (w/v), 0.5% (w/v), 0.51% (w/v), 0.52% (w/v), 0.53% (w/v), 0.54% (w/v), 0.55% (w/v), 0.56% (w/v), 0.57% (w/v), 0.58% (w/v), 0.59% (w/v), 0.6% (w/v), 0.61% (w/v), 0.62% (w/v), 0.63% (w/v), 0.64% (w/v), 0.65% (w/v), 0.66% (w/v), 0.67% (w/v), 0.68% (w/v), 0.69% (w/v), 0.7% (w/v), 0.71% (w/v), 0.72% (w/v), 0.73% (w/v), 0.74% (w/v), 0.75% (w/v), 0.76% (w/v), 0.77% (w/v), 0.78% (w/v), 0.79% (w/v), 0.8% (w/v), 0.81% (w/v), 0.82% (w/v), 0.83% (w/v), 0.84% (w/v), 0.85% (w/v), 0.86% (w/v), 0.87% (w/v), 0.88% (w/v), 0.89% (w/v), 0.9% (w/v), 0.91% (w/v), 0.92% (w/v), 0.93% (w/v), 0.94% (w/v), 0.95% (w/v), 0.96% (w/v), 0.97% (w/v), 0.98% (w/v), 0.99% (w/v), or 1% (w/v) benzalkonium chloride.

In certain embodiments, the absorption enhancer is an alkylsaccharide. In certain embodiments, the alkylsaccharide is chosen from dodecyl maltoside, tetradecyl maltoside (TDM) and sucrose dodecanoate. In certain embodiments, the alkylsaccharide is dodecyl maltoside (the alkylglycoside 1-O-n-dodecyl-β-D-maltopyranoside, alternately referred to as lauryl-β-D-maltopyranoside, dodecyl maltopyranoside, and DDM; C.sub.24H.sub.46Q.sub.11, often referred to by the trade name Intravail®). Alkylsaccharides are used in commercial food and personal care products and have been designated Generally Recognized as Safe (GRAS) substances for food applications. They are non-irritating enhancers of transmucosalabsorption that are odorless, tasteless, non-toxic, non-mutagenic, and non-sensitizing in the Draize test up to a 25% concentration. Alkylsaccharides increase absorption by increasing paracellular permeability, as indicated by a decrease in transepithelial electrical resistance; they may also increase transcytosis. The effect is short-lived. Other alkylsaccharides include tetradecyl maltoside (TDM) and sucrose dodecanoate.

In certain embodiments, an intranasal formulation comprises between about 0.05% (w/v) and about 2.5% (w/v) Intravail®. In certain embodiments, an intranasal formulation comprises between about 0.1% (w/v) and about 0.5% (w/v) Intravail®. In certain embodiments, an intranasal formulation comprises between about 0.15% (w/v) and about 0.35% (w/v) Intravail®. In certain embodiments, an intranasal formulation comprises between about 0.15% (w/v) and about 0.2% (w/v) Intravail®. In certain embodiments, an intranasal formulation comprises about 0.18% (w/v) Intravail®. In certain embodiments, an intranasal formulation comprises about 0.2% (w/v) to about 0.3% (w/v) Intravail®. In certain embodiments, an intranasal formulation comprises about 0.25% (w/v) Intravail®. In certain embodiments, the absorption enhancer is Intravail® (dodecyl maltoside).

In certain embodiments, the pharmaceutical formulation additionally comprises a chelating agent or antioxidant (stabilizing agent) to improve stability. In certain embodiments, the chelating/stabilizing agent is EDTA.

Examples of additional stabilizing agents include: acacia, agar, albumin, alginic acid, aluminum stearate, ammonium alginate, ascorbic acid, ascorbyl palmitate, bentonite, butylated hydroxytoluene (BHT), calcium alginate, calcium stearate, carboxymethylcellulose calcium, carboxymethylcellulose sodium, carrageenan, cellulose, microcrystalline, carboxymethylcellulose sodium, ceratonia, colloidal silicon dioxide, cyclodextrins, diethanolamine, edetates, ethylcellulose, ethylene glycol palmitostearate, glycerin monostearate, guar gum, hectorite, hydroxpropyl betadex, hydroxypropyl cellulose, hypromellose, inulin, invert sugar, lauric acid, lecithin, magnesium aluminum silicate, mineral oil and lanolin alcohols, monoethanolamine, pectin, pentetic acid, phospholipids, polacrilin potassium, poloxamer, polyvinyl alcohol, potassium alginate, potassium chloride, povidone, propyl gallate, propylene glycol, propylene glycol alginate, raffinose, sodium acetate, sodium alginate, sodium borate, sodium stearyl fumarate, sorbitol, stearyl alcohol, sulfobutylether b-cyclodextrin, tagatose, trehalose, triethanolamine, white wax, xanthan gum, xylitol, yellow wax, and zinc acetate.

Examples of additional chelating agents include: citric acid monohydrate, disodium edetate, edetate calcium disodium, edetic acid, fumaric acid, malic acid, maltol, pentetic acid, sodium edetate, and trisodium edetate.

In its capacity as a surfactant, benzalkonium chloride can affect the surface tension of droplets from a delivered nasal spray plume, producing spherical or substantially spherical particles having a narrow droplet size distribution (DSD), as well as the viscosity of a liquid formulation.

In certain embodiments, the absorption enhancer is an alkylsaccharide, for example, a nonionic alkylsaccharide surfactant such as an alkylglycoside and a sucrose ester of fatty acids that consists of an aliphatic hydrocarbon chain coupled to a sugar moiety by a glycosidic or ester bond, respectively. In certain embodiments, the absorption enhancer is an alkylmaltoside (e.g., a tetradecyl maltoside (TDM), a dodecyl maltoside (DDM), etc.). In certain embodiments, the absorption enhancer is sucrose dodecanoate. Alkylsaccharides are used in commercial food and personal care products and have been designated Generally Recognized as Safe (GRAS) substances for food applications. They are non-irritating enhancers of transmucosal absorption that are odorless, tasteless, non-toxic, non-mutagenic, and non-sensitizing in the Draize test up to a 25% concentration. Without being bound to any theory, it is believed that alkylsaccharides increase absorption by increasing paracellular permeability, as indicated by a decrease in transepithelial electrical resistance; they may also increase transcytosis. The effect may be short-lived. In its capacity as an absorption enhancer, alkylmaltosides (e.g., a tetradecyl maltoside (TDM), a dodecyl maltoside (DDM), etc.) can affect the surface tension of droplets from a delivered nasal spray plume, producing spherical or substantially spherical particles having a narrow droplet size distribution (DSD), as well as the viscosity of a liquid formulation.

In certain embodiments, the absorption enhancer is the alkylsaccharide 1-O-n-dodecyl-β-D-maltopyranoside (alternately referred to as lauryl-β-D-maltopyranoside, dodecyl maltopyranoside, dodecyl maltoside, and DDM; C.sub.24H.sub.46Q.sub.11; often referred to by the trade name Intravail®). In certain embodiments, an intranasal formulation comprises about 0.01% (w/v) to about 2.5% (w/v) DDM. In certain embodiments, an intranasal formulation comprises about 0.1% (w/v) to about 0.5% (w/v) DDM. In certain embodiments, an intranasal formulation comprises about 0.15% (w/v) to about 0.35% (w/v) DDM. In certain embodiments, an intranasal formulation comprises about 0.15% (w/v) to about 0.2% (w/v) DDM. In certain embodiments, an intranasal formulation comprises about 0.18% (w/v) DDM. In certain embodiments, an intranasal formulation comprises about 0.2% (w/v) to about 0.3% (w/v) DDM. In certain embodiments, an intranasal formulation comprises about 0.25% (w/v) DDM.

Also provided are nasal drug delivery devices comprising a formulation described herein. In certain embodiments, the device is pre-primed. In certain embodiments, the device can be primed before use. In certain embodiments, the device can be actuated with one hand.

Nasal delivery is considered an attractive, safe, and easy-to-administer route for needle-free, systemic drug delivery, especially when rapid absorption and effect are desired. In addition, nasal delivery may help address issues related to poor bioavailability, slow absorption, drug degradation, and adverse events (AEs) in the gastrointestinal tract and avoids the first-pass metabolism in the liver.

Liquid nasal formulations are mainly aqueous solutions, but suspensions, emulsions, liposomes, and microspheres can also be delivered. Other liquid formulations can comprise liposomes, microspheres, mixed aqueous-organic formulations, non-aqueous formulations, dry powder and retentive formulations (gels). In traditional spray pump systems, antimicrobial preservatives are typically required to maintain microbiological stability in liquid formulations. Metered spray pumps have dominated the nasal drug delivery market since they were introduced. The pumps typically deliver 100 μL (25-250 μL) per spray, and they offer high reproducibility of the emitted dose and plume geometry in in vitro tests.

Examples of standard metered spray pumps include those offered by Aptar Pharma, Inc., such as the multi-dose “classic technology platform” nasal spray devices, and by BD Medical-Pharmaceutical Systems, such as the Accuspray™ system. Such devices comprise a reservoir which holds multiple doses of the nasal spray formulation (e.g., 50, 100, 150, 200, 60, or 120 doses), a closure (e.g., screw, crimp, or snap-on), and an actuator which delivers anywhere from 45 to 1000 μL (e.g., 50, 100, 140, 150, or 200 μL) of fluid per actuation to comprise a single dose. The actuator may be configured to count doses, deliver gel formulations, deliver in an upside-down configuration, etc.

In traditional multi-use spray pump systems, antimicrobial preservatives are typically required to maintain microbiological stability in liquid formulations. However, preservative-free systems are also available, e.g., the Advanced Preservative Free (APF) system from Aptar, which is vented, contains a filter membrane for air flow which prevents contamination, has a metal-free fluid path for oxidizing formulations, and can be used in any orientation. Additional nasal spray devices from Aptar and others are optimized with dispenser tips that prevent clogging (useful for high-viscosity and high-volatile formulations), actuators that do not need re-priming after long periods of disuse, etc. Additional nasal spray devices are propellant driven. Yet additional nasal spray devices include dry powder inhalers.

The particle size and plume geometry can vary within certain limits and depend on the properties of the pump, the formulation, the orifice of the actuator, and the force applied. The droplet size distribution of a nasal spray is a critical parameter, since it significantly influences the in vivo deposition of the drug in the nasal cavity. The droplet size is influenced by the actuation parameters of the device and the formulation. The prevalent median droplet size should be between about 30 and about 100 km. If the droplets are too large (>about 120 km), deposition takes place mainly in the anterior parts of the nose, and if the droplets are too small (<about 10 μm), they can possibly be inhaled and reach the lungs and oral cavity, which should be avoided because of safety reasons. In its capacity as a surfactant, benzalkonium chloride and alkylmaltosides (e.g., a tetradecyl maltoside (TDM), a dodecyl maltoside (DDM), etc.) can affect the surface tension of droplets from a delivered nasal spray plume, producing spherical or substantially spherical particles having a narrow droplet size distribution (DSD), as well as the viscosity of a liquid formulation.

Plume geometry, droplet size and DSD of the delivered plume subsequent to spraying may be measured under specified experimental and instrumental conditions by appropriate and validated and/or calibrated analytical procedures known in the art. These include photography, laser diffraction, and impaction systems (cascade impaction, NGI). Plume geometry, droplet size and DSD can affect pharmacokinetic outcomes such as Cₘₐₓ, Tₘₐₓ, and dose proportionality.

Droplet size distribution can be controlled in terms of ranges for the D10, D50, D90, span [(D90−D10)/D50], and percentage of droplets less than 10 mm. In certain embodiments, the formulation will have a narrow DSD. In certain embodiments, the formulation will have a D(v, 50) of 30-70 μm and a D(v, 90)<100 μm.

In certain embodiments, the percent of droplets less than 10 μm will be less than 10%. In certain embodiments, the percent of droplets less than 10 μm will be less than 5%. In certain embodiments, the percent of droplets less than 10 μm will be less than 2%. In certain embodiments, the percent of droplets less than 10 μm will be less than 1%.

In certain embodiments, the formulation when dispensed by actuation from the device will produce a uniform circular plume with an ovality ratio close to 1. Ovality ratio is calculated as the quotient of the maximum diameter (Dₘₐₓ) and the minimum diameter (Dₘᵢₙ) of a spray pattern taken orthogonal to the direction of spray flow (e.g., from the “top”). In certain embodiments, the ovality ratio is less than ±2.0. In certain embodiments, the ovality ratio is less than ±1.5. In certain embodiments, the ovality ratio is less than ±1.3. In certain embodiments, the ovality ratio is less than ±1.2. In certain embodiments, the ovality ratio is less than ±1.1.

The details and mechanical principles of particle generation for different types of nasal aerosol devices has been described. See, Vidgren and Kublik, Adv. Drug Deliv. Rev. 29:157-77, 1998. Traditional spray pumps replace the emitted liquid with air, and preservatives aretherefore required to prevent contamination. However, driven by the studies suggesting possible negative effects of preservatives, pump manufacturers have developed different spray systems that avoid the need for preservatives. These systems use a collapsible bag, a movable piston, or a compressed gas to compensate for the emitted liquid volume (on the World Wide Web at aptar.com and on the World Wide Web at rexam.com). The solutions with a collapsible bag and a movable piston compensating for the emitted liquid volume offer the additional advantage that they can be emitted upside down, without the risk of sucking air into the dip tube and compromising the subsequent spray. This may be useful for some products where the patients are bedridden and where a head-down application is recommended. Another method used for avoiding preservatives is that the air that replaces the emitted liquid is filtered through an aseptic air filter. In addition, some systems have a ball valve at the tip to prevent contamination of the liquid inside the applicator tip. More recently, pumps have been designed with side-actuation. The pump was designed with a shorter tip to avoid contact with the sensitive mucosal surfaces. New designs to reduce the need for priming and re-priming, and pumps incorporating pressure point features to improve the dose reproducibility and dose counters and lock-out mechanisms for enhanced dose control and safety are available (on the World Wide Web at rexam.com and on the World Wide Web at aptar.com).

Traditional, simple single, bi-dose and multi-use metered-dose spray pumps require priming and some degree of overfill to maintain dose conformity for the labeled number of doses. They are well suited for drugs to be administered daily over a prolonged duration, but due to the priming procedure and limited control of dosing, unless a specialty device is selected, they are less suited for drugs with a narrow therapeutic window of time in which to use the device, particularly if they are not used often. For expensive drugs and drugs intended for single administration or sporadic use and where tight control of the dose and formulation is of importance, single-dose (UDS) or bi-dose spray (BDS) devices are preferred (on the World Wide Web at aptar.com). A simple variant of a single-dose spray device (MAD™) is offered by LMA (LMA, Salt Lake City, Utah, USA; on the World Wide Web at lmana.com). A nosepiece with a spray tip is fitted to a standard syringe. The liquid drug to be delivered is first drawn into the syringe and then the spray tip is fitted onto the syringe. This device has been used in academic studies to deliver, for example, a topical steroid in patients with chronic rhinosinusitis and in a vaccine study. A pre-filled device based on the same principle for one or two doses (Accuspray™, Becton Dickinson Technologies, Research Triangle Park, N.C., USA; on the World Wide Web at bdpharma.com) is used to deliver the influenza vaccine FluMist™ (on the World Wide Web at flumist.com), approved for both adults and children in the US market. A similar device for two doses was marketed by a Swiss company for delivery of another influenza vaccine a decade ago.

Pre-primed single- and bi-dose devices are also available, and consist of a reservoir, a piston, and a swirl chamber (see, e.g., the UDS UnitDose™ and BDS BiDose™ devices from Aptar, formerly Pfeiffer). The spray is formed when the liquid is forced out through the swirl chamber. These devices are held between the second and the third fingers with the thumb on the actuator. A pressure point mechanism incorporated in some devices secures reproducibility of the actuation force and emitted plume characteristics. Currently, marketed nasal migraine drugs like Imitrex® (on the World Wide Web at gsk.com) and Zomig® (on the World Wide Web at az.com; Pfeiffer/Aptar single-dose device), the marketed influenza vaccine Flu-Mist (on the World Wide Web at flumist.com; Becton Dickinson single-dose spray device), and theintranasal formulation of naloxone for opioid overdose rescue, Narcan Nasal® (on the World Wide Web at narcan.com; Adapt Pharma) are delivered with this type of device.

In certain embodiments, the 90% confidence interval for dose delivered per actuation is ±about 2%. In certain embodiments, the 95% confidence interval for dose delivered per actuation is ±about 2.5%.

Historically, intranasal administration of drugs in large volume, such as from syringes adapted with mucosal atomizer devices (MADs), has encountered difficulty due to the tendency of some of the formulation to drip back out of the nostril or down the nasopharynx. Accordingly, in certain embodiments, upon nasal delivery of said pharmaceutical formulation to said patient, less than about 20% of said pharmaceutical formulation leaves the nasal cavity via drainage into the nasopharynx or externally. In certain embodiments, upon nasal delivery of said pharmaceutical formulation to said patient, less than about 10% of said pharmaceutical formulation leaves the nasal cavity via drainage into the nasopharynx or externally. In certain embodiments, upon nasal delivery of said pharmaceutical formulation to said patient, less than about 5% of said pharmaceutical formulation leaves the nasal cavity via drainage into the nasopharynx or externally.

Current container closure system designs for inhalation spray drug products include both pre-metered and device-metered presentations using mechanical or power assistance and/or energy from patient inspiration for production of the spray plume. Pre-metered presentations contain previously measured doses or a dose fraction in some type of units (e.g., single or multiple blisters or other cavities) that are subsequently inserted into the device during manufacture or by the patient before use. Typical device-metered units have a reservoir containing formulation sufficient for multiple doses that are delivered as metered sprays by the device itself when activated by the patient.

With aseptic techniques, the use of preservatives may not be required in pre-primed devices, but overfill is required resulting in a waste fraction similar to the metered-dose, multi-dose sprays. To emit 100 μL, a volume of 125 μL is filled in the device (Pfeiffer/Aptar single-dose device) used for the intranasal migraine medications Imitrex™ (sumatriptan) and Zomig™ (zolmitriptan) and about half of that for a bi-dose design. Sterile drug products may be produced using aseptic processing or terminal sterilization. Terminal sterilization usually involves filling and sealing product containers under high-quality environmental conditions. Products are filled and sealed in this type of environment to minimize the microbial and particulate content of the in-process product and to help ensure that the subsequent sterilization process is successful. In most cases, the product, container, and closure have low bioburden, but they are not sterile. The product in its final container is then subjected to a sterilization process such as heat, irradiation, or chemical (gas). In an aseptic process, the drug product, container, and closure are first subjected to sterilization methods separately, as appropriate, and then brought together. Because there is no process to sterilize the product in its final container, it is critical that containers be filled and sealed in an efficient quality environment. Aseptic processing involves more variables than terminal sterilization. Before aseptic assembly into a final product, the individual parts of the final product generally can be subjected to various sterilization processes. For example, glass containers are subjected to dry heat; rubber closures are subjected to moist heat; and liquid dosage forms are subjected to filtration. Each of these manufacturing processes requires validation and control.

Devices recited herein may employ any of the pharmaceutical formulations, and are useful in the methods disclosed herein.

Accordingly, provided herein are devices adapted for nasal delivery of a pharmaceutical formulation to a patient, comprising a reservoir with a therapeutically effective amount of the formulation hereof.

In certain embodiments, the volume of the pharmaceutical formulation in the reservoir is not more than about 140 μL. In certain embodiments, the volume of the pharmaceutical formulation in the reservoir is above about 125 μL and less than 140 μL. In certain embodiments, about 100 μL of the pharmaceutical formulation in the reservoir is delivered to the patient in one actuation.

In some embodiments, about 100 μL of the pharmaceutical formulation in the reservoir is delivered to the patient in one actuation and comprises less than about 2.5 mg of silver. In some embodiments, about 100 μL of the pharmaceutical formulation in the reservoir is delivered to the patient in one actuation and comprises about 0.5 mg to about 2.5 mg of silver. In some embodiments, about 100 μL of the pharmaceutical formulation in the reservoir is delivered to the patient in one actuation and comprises about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.1 mg, about 2.2 mg, about 2.3 mg, about 2.4 mg, or about 2.5 mg of silver.

In certain embodiments, the pharmaceutical formulation further comprises one or more excipients selected from water, EDTA, and sodium chloride.

In some embodiments, about 100 μL of the aqueous pharmaceutical formulation in the reservoir is delivered to the patient in one actuation.

In certain embodiments, the device is filled with the pharmaceutical formulation using sterile filling.

In certain embodiments, the pharmaceutical formulation is chemically storage-stable for about twelve months at about 25° C. and about 60% relative humidity and about six months at about 40° C. and about 75% relative humidity.

In some embodiments, the composition is delivered with an atomizer. In some embodiments, the atomizer is a handheld battery-driven atomizer intended for nasal drug delivery. In some embodiments, the atomizer atomizes liquids by producing a vortical flow on the droplets as they exit the device. Such devices include the ViaNase™ atomizer (by Kurve Technology Inc., Lynnwood, Wash., USA). In some embodiments, the atomizer is a nasal atomizer driven by highly pressurized nitrogen gas.

In some embodiments, composition is delivered with a nasal powder device. In some embodiments, the nasal powder device is a nasal powder inhaler, nasal powder sprayer, or nasal powder insufflator. Powder sprayers typically have a compressible compartment to provide a pressure that when released creates a plume of powder particles fairly similar to that of a liquid spray. Breath-actuated inhalers require the user to use his or her own breath to inhale the powder into the nostril from a blister or capsule. Nasal insufflator devices consist of a mouthpiece and a nosepiece that are fluidly connected. Delivery occurs when the subject exhales into the mouthpiece to close the velum, and the airflow carries the powder particles into the nose through the device nosepiece.

In some embodiments, the nasal powder inhaler is a blister based powder inhaler.

Typically, the blister is pierced before use and the device nosepiece placed into one nostril. The subject closes the other nostril with the finger and inhales the powder into the nose.

Representative devises include BiDose™/Prohaler™, and Twin-lizer™. Representative nasal powder sprayers include, but are not limited to, UnidoseDP™, Fit-lizer™, Monopowder™ SoluVent™)

It is therefore an object to provide a pharmaceutically acceptable formulation comprising: xylitol; silver nanoparticles; glycyrrhizin; and quercetin. The formulation may be an oral or intranasal liquid. The xylitol may be present in an amount of between 1-10 g/100 ml. The silver nanoparticles may be present in an amount of between 0.001-0.020 g/100 ml. The glycyrrhizin may be present as glycyrrhizic acid in an amount of between 0.1-0.5 g/100 ml. The quercetin may be present in an amount of between 0.1-5 g/100 ml.

The pharmaceutically acceptable formulation may be an oral or intranasal liquid. The xylitol may be present in an amount of at least 1 g/100 ml. The silver nanoparticles may be present in an amount of at least 0.001 g/100 ml. The glycyrrhizin may be present as glycyrrhizic acid in an amount of at least 0.1 g/100 ml. The quercetin may be present in an amount of at least 0.1 g/100 ml.

The formulation may further comprise: ascorbic acid; grapefruit seed extract; monolaurin; eucalyptol; emodin or aloe emodin; zinc citrate; and quinine. The pharmaceutically acceptable formulation may further comprise ascorbic acid in an amount of between 0.1-3 g/100 ml. The pharmaceutically acceptable formulation may further comprise grapefruit seed extract in an amount of between 0.1-0.5 g/100 ml. The pharmaceutically acceptable formulation may further comprise monolaurin in an amount of between 0.1-0.5 g/100 ml. The pharmaceutically acceptable formulation may further comprise eucalyptol in an amount of between 0.10-0.5 g/100 ml. The pharmaceutically acceptable formulation may further comprise emodin in an amount of between 0.01-0.9 g/100 ml. The pharmaceutically acceptable formulation may further comprise aloe emodin in an amount of between 0.01-0.9 g/100 ml.

The pharmaceutically acceptable formulation may further comprise zinc citrate in an amount of between 1-3 g/100 ml. The pharmaceutically acceptable formulation may further comprise quinine in an amount of between 0.005-0.2 g/100 ml.

It is another object to provide a method for reducing the transmissivity of an airborne bacteria or virus, comprising intranasally administering the pharmaceutically acceptable formulation discussed above to a subject.

The subject may be diagnosed to determine infection of the subject with a virus, a coronavirus, SARS-Cov2, SARS-Cov1, MERS, rhinovirus, an influenza virus, and/or bacteria.

The pharmaceutically acceptable formulation may be administered in an oral or intranasal delivery form.

It is another object to provide a pharmaceutical formulation, comprising a solution containing xylitol, 1-20 ppm silver in a silver protein complex and a glycyrrhizin and quercetin complex.

A further object provides a pharmaceutical formulation in a light-proof multidose container, each dose comprising: 1-15 mg soluble oleoresin turmeric, 1-10% xylitol, 0.1-0.5% glycyrrhizic acid, 0.1-5% quercetin, 1-30 mg ascorbic acid, 0.1-0.5% grapefruit seed extract, 0.1-0.5% monolaurin, 0.01-0.5% eucalyptol, 0.01-0.9% emodin or aloe emodin, 1-3% zinc citrate, 5-200 ppm quinine, At least one of potassium sorbate and benzalkonium chloride as a preservative, and 1-20% ethanol, in an aqueous delivery system. The pharmaceutical formulation may further comprise 1-20 μg silver nanoparticles per unit dose. The pharmaceutical formulation may further comprise thymol, a pharmaceutically acceptable flavoring agent, the aqueous delivery system being adjusted to pH 3.0 to pH 4.5.

The present invention provides methods for treating viral infections, comprising intraorally spraying the formulation. The present invention also provides methods for disinfecting respirators, comprising spraying the formulation on a respirator surface.

Methods for prophylactic and anti-transmissivity uses of an anti-microbial composition are provided. The methods comprise the step of administering to a human, an amount of a composition having a first ingredient being xylitol; a second ingredient comprising a glycyrrhizin-protargin-quercetin complex, in addition to formula stabilizing ingredients of glycerol monolaurate, grapefruit seed extract; vitamin C (ascorbic acid), aloe emodin, curcumin, 1,8-cineole, zinc, quinine, flavoring, and an acceptable preservative for use in an oral application. When administered the composition is effective in reducing the incidence of contracting an illness or to prophylactically help prevent transmission of an illness into the or cavity and respiratory tract.

### DETAILED DESCRIPTION OF THE PREFERRED EMBODIMENTS

The invention will be further illustrated by the examples which are not to be construed as limiting the invention in any way. The scope of the invention is to be determined by the claims appended hereto.

### Example 1

A formulation is provided in a purified water solution, 100 ml:

A composition is prepared for a dilute bathing formulation of xylitol for nasal delivery.

The concentration of xylitol in the solution may be 10%-60% by weight/volume. This corresponds to 10-60 grams of xylitol per 100 ml of solution. xylitol is soluble in solution up to 64.2 grams per 100 ml of solution.

The following components were used to prepare the nasal delivery composition of Example 1:

TABLE 1

| COMPONENTS | AMOUNT (g/100 ml) |
|---|---|
| Xylitol | 1-10 |
| Silver nanoparticles | 0.001-0.020 |
| (complexed with protein) | |
| Benzalkonium Chloride (50%), N.F. | 0.04 |
| Or | |
| Potassium sorbate | 0.1-0.4 |
| Soluble oleoresin turmeric | 0.1-1.5 |
| Glycyrrhizic acid | 0.1-0.5 |
| Quercetin | 0.1-5 |
| Ascorbic acid | 0.1-3 |
| Grapefruit seed extract | 0.1-0.5 |
| Monolaurin | 0.1-0.5 |
| Eucalyptol | 0.01-0.5 |
| Emodin or aloe emodin | 0.01-0.9 |
| Zinc citrate | 1-3 |
| Quinine | 0.005-0.2 |
| Purified Water, U.S.P. q.s. | 100 ml |

In addition, a citrate buffer and HCl or NaOH to adjust pH to 3.0, 5.0, 7.0, 7.4 or 7.8 may be employed.

An oral formulation may be provided in an aqueous delivery system with an appropriate preservative system using potassium sorbate and/or benzalkonium chloride with 1-20% ethanol, thymol, and an acceptable flavoring at an adjusted pH of 3.0 to 4.5. This may be delivered as an oral spray and/or as a treatment for the inside of a facial mask

The dosage range used for the formulation is anywhere from 0.1-1 ml per dose. The formulation may be administered 1-4 times daily. Depending on the amounts of xylitol added to the solution a 0.1 ml dose would therefore deliver anywhere from 10-60 mgs of xylitol.

Accordingly, a 0.4 ml dose of the nasal spray would administer anywhere from 4-240 mgs of xylitol. Likewise, the 0.1 ml dose of spray would deliver up to 20 μg silver.

A composition of Example 1 consists, e.g., of xylitol, silver, glycyrrhizic acid, and quercetin, which are the main active ingredients in the medicament, a buffer system, (consisting of sodium citrate dihydrate, citric acid anhydrous, and hydrochloric acid or sodium hydroxide), as well as an antimicrobial preservative, benzalkonium chloride (50%), all dissolved in 100 ml of water, along with the soluble oleoresin turmeric, ascorbic acid, grapefruit seed extract, monolaurin, eucalyptol, emodin or aloe emodin, zinc citrate and quinine.

A simplified embodiment is shown in Table 2:

TABLE 2

| COMPONENTS | AMOUNT (g/100 ml) |
|---|---|
| Xylitol | 1-10 |
| Silver nanoparticles | 0.001-0.020 |
| (complexed with protein) | |
| Glycyrrhizic acid | 0.1-0.5 |
| Quercetin | 0.1-5 |
| Purified Water, U.S.P. q.s. | 100 ml |

An embodiment with a higher silver level is shown in in Table 3:

TABLE 3

| COMPONENTS | AMOUNT (g/100 ml) |
|---|---|
| Xylitol | 1-10 |
| Silver nanoparticles | 0.01-0.05 |
| (complexed with protein or quercetin) | |
| Benzalkonium Chloride (50%), N.F. | 0.04 |
| Glycyrrhizic acid | 0.1-0.5 |
| Quercetin | 0.1-5 |
| Ascorbic acid | 0.1-3 |
| Eucalyptol | 0.01-0.5 |
| Emodin or aloe emodin | 0.01-0.9 |
| Zinc citrate | 1-3 |
| Quinine | 0.005-0.2 |
| Purified Water, U.S.P. q.s. | 100 ml |

### OTHER EMBODIMENTS

Also provided are embodiments wherein any embodiment above can be combined with any one or more of these embodiments, provided the combination is not mutually exclusive. Also provided herein are uses in the treatment of indications or one or more symptoms thereof as disclosed herein, and uses in the manufacture of medicaments for the treatment of indications or one or more symptoms thereof as disclosed herein, equivalent in scope to any embodiment disclosed above, or any combination thereof that is not mutually exclusive. The methods and uses may employ any of the devices disclosed herein, or any combination thereof that is not mutually exclusive, or any of the pharmaceutical formulations disclosed herein, or any combination thereof that is not mutually exclusive.

Although the present invention has been described with reference to specific details of certain embodiments thereof in the above examples, it will be understood that modifications and variations are encompassed within the spirit and scope of the invention.

### REFERENCES (EACH REFERENCE CITED HEREIN IS EXPRESSLY INCORPORATED HEREIN BY REFERENCE IN ITS ENTIRETY)

2014; 58(11):6970-6973. doi:10.1128/AAC.03672-14
Abd-Alla H I, Abu-Gabal N S, Hassan A Z, El-Safty M M, Shalaby N M. Antiviral activity of Aloe hijazensis against some haemagglutinating viruses infection and its phytoconstituents. Arch Pharm Res. 2012; 35(8):1347-1354. doi:10.1007/s12272-012-0804-5
Akram M Tahir I M Shah S M A Mahmood Z Altaf A Antiviral potential of medicinal plants against HIV, HSV, influenza, hepatitis, and coxsackievirus: a systematic review. Phytotherapy Research. 2018; 32:811.
Andrighetti-Fröhner C Sincero T C M Da Silva A C Savi L A Gaido C M Antiviral evaluation of plants from Brazilian Atlantic tropical forest. Fitoterapia. 2005; 76:374.—PubMed
Arbab A H Parvez M K Al Dosari M S Al Rehaily A J In vitro evaluation of novel antiviral activities of 60 μmedicinal plants extracts against hepatitis B virus. Experimental and Therapeutic Medicine. 2017; 14:626.
Arch Pharm Res. 2012; 35(8):1347-1354. doi:10.1007/s12272-012-0804-5
Asres K Bucar F Anti-HIV activity against immunodeficiency virus type 1 (HIV-I) and type II (HIV-II) of compounds isolated from the stem bark of Combretum molle. Ethiopian Medical Journal. 2005; 43:15.
Badam L. In vitro antiviral activity of indigenous glycyrrhizin, licorice and glycyrrhizic acid (Sigma) on Japanese encephalitis virus. J Commun Dis. 1997; 29(2):91-99.
Baraboi, et al., “Mechanism of the antistressor and antiradiation action of plant phenol compounds”, Ukr Biokhim Zh November-December 1998; 70(6):13-23 Abstract.
Ben-Shabat S Yarmolinsky L Porat D Dahan A Antiviral effect of phytochemicals from medicinal plants: applications and drug delivery strategies. Drug Delivery and Translational Research. 2020; 10:354.
Braga, Lilian R., Leonardo M. Pérez, Marina del V. Soazo, and Fabricio Machado. “Evaluation of the antimicrobial, antioxidant and physicochemical properties of Poly (Vinyl chloride) films containing quercetin and silver nanoparticles.” Lwt 101 (2019): 491-498.
Brouet et al., “Curcumin an anti-tumour promoter and anti-inflammatory agent, inhibits induction of nitric oxide synthase in activated macrophages”, Biochem Biophys Res CommunJan. 17, 1995; 206.
Bruno Rafael Pereira Lopes, Mirian Feliciano da Costa, Amanda Genova Ribeiro, Tiago Francisco da Silva, Caroline Sprengel Lima, Icaro Putinhon Caruso, Gabriela Campos de Araujo, Leticia Hiromi Kubo, Federico Iacovelli, Mattia Falconi, Alessandro Desideri, Juliana de Oliveira, Luis Octavio Regasini, Fatima Pereira de Souza, Karina Alves Toledo Quercitin Pentaacetate Inhibits in Vitro Human Respiratory Syncytial Virus Adhesion. Virus Res, 276, 197805, 2020 Jan. 15
Channappanavar R, Perlman S. Pathogenic human coronavirus infections: causes and consequences of cytokine storm and immunopathology. Semin Immunopathol. 2017; 39(5):529-539. doi:10.1007/s00281-017-0629-x
Ciavarella C, Motta I, Valente S, Pasquinelli G. Pharmacological (or Synthetic) and Nutritional Agonists of PPAR-7 as Candidates for Cytokine Storm Modulation in COVID-19 Disease. Molecules. 2020; 25(9):2076. Published 2020 Apr. 29. doi:10.3390/molecules25092076
Cinatl J, Morgenstern B, Bauer G, et al. Glycyrrhizin, an active component of liquorice roots, and replication of SARS-associated coronavirus. Lancet. 2003 Jun. 14; 361(9374):2045-6. doi: 10.1016/s0140-6736(03)13615-x. PMID: 12814717
Clarke N M, May J T. Effect of antimicrobial factors in human milk on rhinoviruses and milk-borne cytomegalovirus in vitro. J Med Microbiol. 2000; 49(8):719-723. doi:10.1099/0022-1317-49-8-719
Cort W M. Antioxidant activity of tocopherols, ascorbyl palmitate, and ascorbic acid and their mode of action. J Am Oil Chem Soc. 1974 July; 51(7):321-5. doi: 10.1007/bf02633006. PMID: 4845640
Cross, Karen J., et al, Mechanisms of cell by influenza virus, Expert News in Molecular Medicine: www.ermm.cbcu.cam.ac.uk. Aug. 6, 2001.
Dai J P, Wang Q W, Su Y, et al. Emodin Inhibition of Influenza A Virus Replication and Influenza Viral Pneumonia via the Nrf2, TLR4, p38/JNK and NF-kappaB Pathways. Molecules. 2017; 22(10):1754. Published 2017 Oct. 18. doi:10.3390/molecules22101754
Deshpande D A, Wang W C, McIlmoyle E L, et al. Bitter taste receptors on airway smooth muscle bronchodilate by localized calcium signaling and reverse obstruction. Nat Med. 2010; 16(11):1299-1304. doi:10.1038/nm.2237
Ding Y, Xu J, Cheng L B, et al. Effect of Emodin on Coxsackievirus B3 μm-Mediated Encephalitis in Hand, Foot, and Mouth Disease by Inhibiting Toll-Like Receptor 3 Pathway In Vitro and In Vivo. J Infect Dis. 2020; 222(3):443-455. doi:10.1093/infdis/jiaa093
Duarte, et al., “Vasodilator effects of quercetin in isolated rat vascular smooth muscle”, Eur J Pharmacol August 1993 239:1-7
Duke, et al., “Biological Activities of CURCUMINOIDS”, Phytochemical and Ethnobotanical Database, 2003.
Elena E Paskaleva 1, Jing Xue, David Y-W Lee, Alexander Shekhtman, Mario Canki. Palmitic Acid Analogs Exhibit Nanomolar Binding Affinity for the HIV-1 CD4 Receptor and Nanomolar Inhibition of gp120-to-CD4 Fusion. PLoS One 5 (8), e12168 2010 August
Fiore C, Eisenhut M, Krausse R, et al. Antiviral effects of Glycyrrhiza species. Phytother Res. 2008; 22(2):141-148. doi:10.1002/ptr.2295
Frasnelli J, Gingras-Lessard F, Robert J, Steffener J. The Effect of Stimulus Duration on the Nostril Localization of Eucalyptol. Chem Senses. 2017; 42(4):303-308. doi:10.1093/chemse/bjx008
Ge X Y, Li J L, Yang X L, et al. Isolation and characterization of a bat SARS-like coronavirus that uses the ACE2 receptor. Nature. 2013; 503(7477):535-538. doi:10.1038/nature12711
Gondim F L, Serra D S, Cavalcante F S Á. Effects of Eucalyptol in respiratory system mechanics on acute lung injury after exposure to short-term cigarette smoke. Respir Physiol Neurobiol. 2019; 266:33-38. doi:10.1016/j.resp.2019.04.007
Grassin-Delyle S, Abrial C, Fayad-Kobeissi S, et al. The expression and relaxant effect of bitter taste receptors in human bronchi. Respir Res. 2013; 14(1):134. Published 2013 Nov. 22. doi:10.1186/1465-9921-14-134
Grishko V V, Galaiko N V, Tolmacheva I A, et al. Functionalization, cyclization and antiviral activity of A-secotriterpenoids. Eur J Med Chem. 2014; 83:601-608. doi:10.1016/j.ejmech.2013.12.058
Haase A T, Rakasz E, Schultz-Darken N, et al. Glycerol Monolaurate Microbicide Protection against Repeat High-Dose SIV Vaginal Challenge. PLoS One. 2015; 10(6):e0129465. Published 2015 Jun. 9. doi:10.1371/journal.pone.0129465
Hamming I, Timens W, Bulthuis M L, Lely A T, Navis G, van Goor H. Tissue distribution of ACE2 protein, the functional receptor for SARS coronavirus. A first step in understanding SARS pathogenesis. J Pathol. 2004; 203(2):631-637. doi:10.1002/path.1570
Hammock B D, Wang W, Gilligan M M, Panigrahy D. Eicosanoids: The Overlooked Storm in Coronavirus Disease 2019 (COVID-19)? [published online ahead of print, 2020 Jul. 8]. Am J Pathol. 2020; 50002-9440(20)30332-1. doi:10.1016/j.ajpath.2020.06.010
Hardy M E, Hendricks J M, Paulson J M, Faunce N R. 180-glycyrrhetinic acid inhibits rotavirus replication in culture. Virol J. 2012; 9:96. Published 2012 May 22. doi:10.1186/1743-422X-9-96
Hayden, F G, et al., Use of the oral neuraminidase inhibitor oseltamivir in experimental human influenza: randomized controlled trials for prevention and treatment, JAMA. Oct. 6, 1999; 282 (13):1240-6, PubMed, National Library of Medicine, PMID: 10517426, Abstract, 2 pgs.
He S, Lin Q, Qu M, et al. Liver-Targeted Co-delivery of Entecavir and Glycyrrhetinic Acid Based on Albumin Nanoparticle To Enhance the Accumulation of Entecavir. Mol Pharm. 2018; 15(9):3953-3961. doi:10.1021/acs.molpharmaceut.8b00408
Hentschel C, Eglau M C, Hahn E G, Curcuma xanhorrhiza (Java turmeric) in clinicaluse], Fortschr Med. Sep. 30, 1996; 114(27):349-50
Hess D J, Henry-Stanley M J, Wells C L. Antibacterial synergy of glycerol monolaurate and aminoglycosides in Staphylococcus aureus biofilms. Antimicrob Agents Chemother.
Heurich A, Hofmann-Winkler H, Gierer S, Liepold T, Jahn O, Pöhlmann S., TMPRSS2 and ADAM17 cleave ACE2 differentially and only proteolysis by TMPRSS2 augments entry driven by the severe acute respiratory syndrome coronavirus spike protein. J Virol. 2014; 88(2):1293-1307. doi:10.1128/JVI.02202-13
Ho T Y, Wu S L, Chen J C, Li C C, Hsiang C Y. Emodin blocks the SARS coronavirus spike protein and angiotensin-converting enzyme 2 interaction. Antiviral Res. 2007; 74(2):92-101. doi:10.1016/j.antiviral.2006.04.014
Hsiang C Y, Ho T Y. Emodin is a novel alkaline nuclease inhibitor that suppresses herpes simplex virus type 1 yields in cell cultures. Br J Pharmacol. 2008; 155(2):227-235. doi:10.1038/bjp.2008.242
Hlu, Chao-Chin, Wen-Kang Chen, Pel-Hu Liao, Wei-Che Yu, and Yean-Jang Lee. “Synergistic effect of cadmium chloride and acetaldehyde on cytotoxicity and its prevention by quercetin and glycyrrhizin.” Mutation Research/Genetic Toxicology and Environmental Mutagenesis 496, no. 1-2 (2001): 117-1217.
Hussain H, Green I R, Shamraiz U, et al. Therapeutic potential of glycyrrhetinic acids: a patent review (2010-2017). Expert Opin Ther Pat. 2018; 28(5):383-398. doi:10.1080/13543776.2018.1455828
Inoue K, Takano H. Therapeutic effects of inhaled 1,8-cineole on allergic airway inflammation. Basic Clin Pharmacol Toxicol. 2011; 108(5):295-296. doi:10.1111/j.1742-7843.2011.00679.x
Ito M, Nakashima H, Baba M, et al. Inhibitory effect of glycyrrhizin on the in vitro infectivity and cytopathic activity of the human immunodeficiency virus [HIV (HTLV-III/LAV)]. Antiviral Res. 1987; 7(3):127-137. doi:10.1016/0166-3542(87)90001-5
Ito M, Sato A, Hirabayashi K, et al. Mechanism of inhibitory effect of glycyrrhizin on replication of human immunodeficiency virus (HIV). Antiviral Res. 1988; 10(6):289-298. doi:10.1016/0166-3542(88)90047-2
Jahan I, Onay A. Potentials of plant-based substance to inhabit and probable cure for the COVID-19. Turk J Biol. 2020; 44(3):228-241. Published 2020 June 21. doi:10.3906/biy-2005-114
Jhanwar, Bharat, and Somdatt Gupta. “Biopotentiation using herbs: Novel technique for poor bioavailable drugs.” Int J PharmTech Res 6, no. 2 (2014): 443-454.
Jia H P, Look D C, Shi L, et al. ACE2 receptor expression and severe acute respiratory syndrome coronavirus infection depend on differentiation of human airway epithelia. J Virol. 2005; 79(23):14614-14621. doi:10.1128/JVI.79.23.14614-14621.2005
Jiang F, Wu G, Li W, et al. Preparation and protective effects of 1,8-cineole-loaded self-microemulsifying drug delivery system on lipopolysaccharide-induced endothelial injury in mice. Eur J Pharm Sci. 2019; 127:14-23. doi:10.1016/j.ejps.2018.10.012
Jori A. The Sugar Alcohols: A Profile. Advances in Pharmacology and Chemotherapy (1984) 20: 191-218.
Juergens U R, Engelen T, Racké K, Stöber M, Gillissen A, Vetter H. Inhibitory activity of 1,8-cineol (eucalyptol) on cytokine production in cultured human lymphocytes and monocytes. Pulm Pharmacol Ther. 2004; 17(5):281-287. doi:10.1016/j.pupt.2004.06.002
Juergens U R. Anti-inflammatory properties of the monoterpene 1.8-cineole: current evidence for co-medication in inflammatory airway diseases. Drug Res (Stuttg). 2014; 64(12):638-646. doi:10.1055/s-0034-1372609
Kaji, M., Neuraminidase inhibitor, anti-influenzal agent—mechanism of action, and how to use clinically, Nippon Rinsho. November 2003; 61(11):1975-9, PubMed, National Library of Medicine, PMID:14649441, Abstract, 1 pg.
Kannan S, Shaik Syed Ali P, Sheeza A, Hemalatha K. COVID-19 (NovelCoronavirus 2019)—recent trends. Eur Rev Med Pharmacol Sci. 2020; 24(4):2006-2011. doi:10.26355/eurrev_202002_20378
Karamian, Roya, and Mostafa Asadbegy. “Green Biosynthesis of Silver Nanoparticles Using Glycyrrhiza glabra L. Extract and Evaluation of their Selective Antimicrobial Activity.” Organic Chemistry Research 4, no. 2 (2018): 194-209.
Kennedy-Feitosa E, Cattani-Cavalieri I, Barroso M V, Romana-Souza B, Brito-Gitirana L, Valenca S S. Eucalyptol promotes lung repair in mice following cigarette smoke-induced emphysema. Phytomedicine. 2019; 55:70-79.
Khan A, Vaibhav K, Javed H, et al. 1,8-cineole (eucalyptol) mitigates inflammation in amyloid Beta toxicated PC12 cells: relevance to Alzheimer's disease. Neurochem Res. 2014; 39(2):344-352. doi:10.1007/s11064-013-1231-9
Kontiokari T, Uhari M, Koskela, M. Antiadhesive effects of xylitol on otopathogenic bacteria. Jour of Antimicrobial Chemotherapy (1998) 41; 563-565.
Kuba K, Imai Y, Rao S, et al. A crucial role of angiotensin converting enzyme 2 (ACE2) in SARS coronavirus-induced lung injury. Nat Med. 2005; 11(8):875-879. doi:10.1038/nm1267
Lambert D W, Yarski M, Warner F J, et al. Tumor necrosis factor-alpha convertase (ADAM17) mediates regulated ectodomain shedding of the severe-acute respiratory syndrome-coronavirus (SARS-CoV) receptor, angiotensin-converting enzyme-2 (ACE2). J Biol Chem. 2005; 280(34):30113-30119. doi:10.1074/jbc.M505111200
Lee Jia Ming 1, Adeline Chia Yoke Yin. Therapeutic Effects of Glycyrrhizic Acid. Nat Prod Commun., 8 (3), 415-8 Mar. 2013
Li L, Song X, Yin Z, et al. The antibacterial activity and action mechanism of emodin from Polygonum cuspidatum against Haemophilus parasuis in vitro. Microbiol Res. 2016; 186-187:139-145. doi:10.1016/j.micres.2016.03.008
Li S W, Yang T C, Lai C C, et al. Antiviral activity of aloe-emodin against influenza A virus via galectin-3 up-regulation. Eur J Pharmacol. 2014; 738:125-132. doi:10.1016/j.ejphar.2014.05.028
Li W, Moore M J, Vasilieva N, et al. Angiotensin-converting enzyme 2 is a functional receptor for the SARS coronavirus. Nature. 2003; 426(6965):450-454. doi:10.1038/nature02145
Li Y, Lai Y, Wang Y, Liu N, Zhang F, Xu P. 1, 8-Cineol Protect Against Influenza-Virus-Induced Pneumonia in Mice. Inflammation. 2016; 39(4):1582-1593. doi:10.1007/s10753-016-0394-3
Li Y, Xu Y L, Lai Y N, Liao S H, Liu N, Xu P P. Intranasal co-administration of 1,8-cineole with influenza vaccine provide cross-protection against influenza virus infection. Phytomedicine. 2017; 34:127-135. doi:10.1016/j.phymed.2017.08.014
Li, Fang; et al. Structural basis of receptor recognition by SARS-CoV-2. Nature ORCID: orcid.org/0000-0003-2600-60591
Lima P R, de Melo T S, Carvalho K M, et al. 1,8-cineole (eucalyptol) ameliorates cerulein-induced acute pancreatitis via modulation of cytokines, oxidative stress and NF-1B activity in mice. Life Sci. 2013; 92(24-26):1195-1201. doi:10.1016/j.lfs.2013.05.009
Lin C W, Wu C F, Hsiao N W, et al. Aloe-emodin is an interferon-inducing agent with antiviral activity against Japanese encephalitis virus and enterovirus 71. Int J Antimicrob Agents. 2008; 32(4):355-359. doi:10.1016/j.ijantimicag.2008.04.018
Lin, et al., “Recent studies on the biofunctions and biotransformations of curcumin”, Biofactors 2000; 13(1-4):153-8
Liu Z, Ma N, Zhong Y, Yang Z Q. Antiviral effect of emodin from Rheum palmatum against coxsakievirus B5 and human respiratory syncytial virus in vitro. J Huazhong Univ Sci Technolog Med Sci. 2015; 35(6):916-922. doi:10.1007/s11596-015-1528-9
Liu, Jinjun. S. L. T. U. Weiyi, and L. I. Qiangbai. “Anticancer nano-silver composition for treatment of lung cancer, and preparation method and use thereof.” U.S. Pub. Application 2017/0119818; U.S. Ser. No. 15/330,759, filed May 4, 2017.
Martins AOBPB, Rodrigues L B, Cesário FRAS, et al. Anti-edematogenic and anti-inflammatory activity of the essential oil from Croton rhamnifolioides leaves and its major constituent 1,8-cineole (eucalyptol). Biomed Pharmacother. 2017; 96:384-395. doi:10.1016/j.biopha.2017.10.005
Martirosyan, Alina, Konstantinos Grintzalis, Madeleine Polet, Laurie Laloux, and Yves-Jacques Schneider. “Tuning the inflammatory response to silver nanoparticles via quercetin in Caco-2 (co-) cultures as model of the human intestinal mucosa.” Toxicology letters 253 (2016): 36-45.
Matoba Y, Aoki Y, Tanaka S, et al. HeLa-ACE2-TMPRSS2 Cells Are Useful for the Isolation of Human Coronavirus 229E. Jpn J Infect Dis. 2016; 69(5):452-454. doi:10.7883/yoken.JJID.2016.106
Milewska A, Nowak P, Owczarek K, et al. Entry of Human Coronavirus NL63 into the Cell. J Virol. 2018; 92(3):e01933-17. Published 2018 January 17. doi:10.1128/JVI.01933-17
Moghimi R, Aliahmadi A, Rafati H. Ultrasonic nanoemulsification of food grade trans-cinnamaldehyde: 1,8-Cineol and investigation of the mechanism of antibacterial activity. Ultrason Sonochem. 2017; 35(Pt A):415-421. doi:10.1016/j.ultsonch.2016.10.020
Monisha B A, Kumar N, Tiku A B. Emodin and Its Role in Chronic Diseases. Adv Exp Med Biol. 2016; 928:47-73. doi:10.1007/978-3-319-41334-1_3
Mueller E A, Schlievert P M. Non-aqueous glycerol monolaurate gel exhibits antibacterial and anti-biofilm activity against Gram-positive and Gram-negative pathogens. PLoS One. 2015; 10(3):e0120280. Published 2015 Mar. 23. doi:10.1371/journal.pone.0120280
Murata S, Shiragami R, Kosugi C, et al. Antitumor effect of 1, 8-cineole against colon cancer. Oncol Rep. 2013; 30(6):2647-2652. doi:10.3892/or.2013.2763
Murray R K, Granner D K, Mayes P A, Rodwell V W. Harper's Biochemistry. Appleton and Lange; Stamford, Conn. 1996.
Myndi G. Holbrook, B.Sc., Amandine Gamble, Ph.D., Brandi N. Williamson, M.P.H., Azaibi Tamin, Ph.D., Jennifer L. Harcourt, Ph.D., Natalie J. Thornburg, Ph.D., Susan I. Gerber, M.D. Aerosol and Surface Stability of SARS-CoV-2 as Compared with SARS-CoV-1. Mar. 17, 2020. New England Journal of Medicine, 10.1056/NEJMc2004973.
Naaber P, Lehto E, Salminen S, Mikelsaar M. Inhibition of adhesion of Clostridium difficile to Caco-2 cells. FEMS Immunology and Medical Microbiology. (1996) 14; 205-209.
Nafisi S, Bonsaii M, Manouchehri F, Abdi K. Interaction of glycyrrhizin and glycyrrhetinic acid with DNA. DNA Cell Biol. 2012; 31(1):114-121. doi:10.1089/dna.2011.1287 Neeltje van Doremalen, Ph.D., Trenton Bushmaker, B.Sc., Dylan H. Morris, M.Phil.,
Nikolić B, Mitić-Culafić D, Vuković-Gačić B, Knežević-Vukčević J. Modulation of genotoxicity and DNA repair by plant monoterpenes camphor, eucalyptol and thujone in Escherichia coli and mammalian cells. Food Chem Toxicol. 2011; 49(9):2035-2045. doi:10.1016/j.fct.2011.05.015
Nikolic I, Mitsou E, Pantelic I, et al. Microstructure and biopharmaceutical performances of curcumin-loaded low-energy nanoemulsions containing eucalyptol and pinene: Terpenes' role overcome penetration enhancement effect?. Eur J Pharm Sci. 2020; 142:105135. doi:10.1016/j.ejps.2019.105135
Nkoghe, D et al., Influenza: from vaccine prevention to antiviral therapy, Rev Med Liege. 2000; 55(1):19-23, PubMed, National Library of Medicine, PMID: 10803033,
On, et al., “Vitamin c prevents radiation-induced endothelium-dependent vasomotor dysfunction and de-endothelialization by inhibiting oxidative damage in the rat”, Clin Exp Pharmacol Physiol October 2001; 28(10):816-21
Oudit G Y, Kassiri Z, Jiang C, et al. SARS-coronavirus modulation of myocardial ACE2 expression and inflammation in patients with SARS. Eur J Clin Invest. 2009; 39(7):618-625. doi:10.1111/j.1365-2362.2009.02153.x
Parvez M K, Al-Dosari M S, Alam P, Rehman M, Alajmi M F, Algahtani A S. The anti-hepatitis B virus therapeutic potential of anthraquinones derived from Aloe vera. PhytotherRes. 2019; 33(11):2960-2970. doi:10.1002/ptr.6471
Pompei R, Flore O, Marccialis M A, Pani A, Loddo B. Glycyrrhizic acid inhibits virus growth and inactivates virus particles. Nature. 1979; 281(5733):689-690. doi:10.1038/281689a0
Pompei R, Pani A, Flore O, Marcialis M A, Loddo B. Antiviral activity of glycyrrhizic acid. Experientia. 1980; 36(3):304. doi:10.1007/BF01952290
Račková, Lucia, Viera Jančinová, Margita Petríková, Katarína Drábiková, Radomír Nosál', Milan Štefek, Daniela Košt'lová, Nad'a Prónayová, and Maria Kováčová. “Mechanism of anti-inflammatory action of liquorice extract and glycyrrhizin.” Natural product research 21, no. 14 (2007): 1234-1241.
Ragab D, Salah Eldin H, Taeimah M, Khattab R, Salem R. The COVID-19 Cytokine Storm; What We Know So Far. Front Immunol. 2020; 11:1446. Published 2020 Jun. 16. doi:10.3389/fimmu.2020.01446
Robak, et al., “Bioactivity of flavonoids”, Pol J Pharmacol November-December 1996; 48(6):555-64
Robson B. COVID-19 Coronavirus spike protein analysis for synthetic vaccines, a peptidomimetic antagonist, and therapeutic drugs, and analysis of a proposed achilles' heel conserved region to minimize probability of escape mutations and drug resistance. Comput Biol Med. 2020; 121:103749. doi:10.1016/j.compbiomed.2020.103749
Rodenak-Kladniew B, Castro A, Starkel P, Galle M, Crespo R. 1,8-Cineole promotes G0/G1 cell cycle arrest and oxidative stress-induced senescence in HepG2 cells and sensitizes cells to anti-senescence drugs. Life Sci. 2020; 243:117271. doi:10.1016/j.lfs.2020.117271 Rogero M M, Leão M C, Santana T M, et al. Potential benefits and risks of omega-3 fatty acids supplementation to patients with COVID-19 [published online ahead of print, 2020 Jul. 10]. Free Radic Biol Med. 2020; 156:190-199. doi:10.1016/j.freeradbiomed.2020.07.005
Sampath S, Veeramani V, Krishnakumar G S, Sivalingam U, Madurai S L, Chellan R. Evaluation of in vitro anticancer activity of 1,8-Cineole-containing n-hexane extract of Callistemon citrinus (Curtis) Skeels plant and its apoptotic potential. Biomed Pharmacother. 2017; 93:296-307. doi:10.1016/j.biopha.2017.06.056
Schlievert P M, Kilgore S H, Kaus G M, Ho T D, Ellermeier C D. Glycerol Monolaurate (GML) and a Nonaqueous Five-Percent GML Gel Kill Bacillus and Clostridium Spores. mSphere. 2018; 3(6):e00597-18. Published 2018 Nov. 21. doi:10.1128/mSphereDirect.00597-18 Schlievert P M, Kilgore S H, Seo K S, Leung D Y M. Glycerol Monolaurate Contributes to the Antimicrobial and Anti-inflammatory Activity of Human Milk. Sci Rep. 2019; 9(1):14550. Published 2019 Oct. 10. doi:10.1038/s41598-019-51130-y
Schwarz S, Wang K, Yu W, Sun B, Schwarz W. Emodin inhibits current through SARS-associated coronavirus 3a protein. Antiviral Res. 2011; 90(1):64-69. doi:10.1016/j.antiviral.2011.02.008
Sen'kova A V, Warszycki D, Salomatina O V, Salakhutdinov N F, Zenkova M A, Logashenko E B. Soloxolone methyl inhibits influenza virus replication and reduces virus-induced lung inflammation. Sci Rep. 2017 Oct. 25; 7(1):13968. doi: 10.1038/s41598-017-14029-0. PMID: 29070858
Shobana, et al., “Antioxidant activity of selected Indian spices”, Prostaglandins Leukot Essent Fatty Acids February 2000; 62(2):107-10
Shuangsuo D, Zhengguo Z, Yunru C, et al. Inhibition of the replication of hepatitis B virus in vitro by emodin. Med Sci Monit. 2006; 12(9):BR302-BR306.
Skaper, et al., “Quercetin protects cutaneous tissue-associated cell types including sensory neurons from oxidative stress induced by glutathione depletion: cooperative effects of ascorbic acid”, Free Radic Biol Med 1997; 22(4):669-78 Abstract.
Smart R C, Crawford C L. Effect of ascorbic acid and its synthetic lipophilic derivative ascorbyl palmitate on phorbol ester-induced skin-tumor promotion in mice. Am J Clin Nutr. 1991 December; 54(6 Suppl):12665-12735. doi: 10.1093/ajcn/54.6.1266s. PMID: 1962581
Sobreira Dantas Nóbrega de Figuêiredo F R, Monteiro Á B, Alencar de Menezes I R, et al. Effects of the Hyptis martiusii Benth. leaf essential oil and 1,8-cineole (eucalyptol) on the central nervous system of mice. Food Chem Toxicol. 2019; 133:110802. doi:10.1016/j.fct.2019.110802 Song W, Gui M, Wang X, Xiang Y. Cryo-EM structure of the SARS coronavirus spike glycoprotein in complex with its host cell receptor ACE2. PLoS Pathog. 2018; 14(8):e1007236. Published 2018 Aug. 13. doi:10.1371/journal.ppat.1007236
Sordillo P P, Helson L. Curcumin suppression of cytokine release and cytokine storm. A potential therapy for patients with Ebola and other severe viral infections. In Vivo. 2015; 29(1):1-4.
Speshock, Janice and Hussain, Saber. Novel Nanotechnology-Based Antiviral Agents, Applied Biotechnology Branch 711th Human Performance Wing, Air Force Research.
Stanetty C, Wolkerstorfer A, Amer H, et al. Synthesis and antiviral activities of spacer-linked 1-thioglucuronide analogues of glycyrrhizin. Beilstein J Org Chem. 2012; 8:705-711. doi:10.3762/bjoc.8.79
Steer S A, Corbett J A, The role and regulation of COX-2 during viral infection, Viral Immunol. 2003; 16(4):447-60
Stromqvist M, Falk P, Bergstrom S, Hannson L, Lonnerdal B, Normark S, and Hernell O. Human milk kappa-casein and inhibition of Helicobacter pylori adhesion in human gastric mucosa. J Pediatr Gastroenterol Nutr. (October 1995); 21(3): 288-96.
Sun X, Wang T, Cai D, et al. Cytokine storm intervention in the early stages of COVID-19 pneumonia. Cytokine Growth Factor Rev. 2020; 53:38-42. doi:10.1016/j.cytogfr.2020.04.002
Sun, Dongdong, Weiwei Zhang, Zhipeng Mou, Ying Chen, Feng Guo, Endong Yang, and Weiyun Wang. “Transcriptome analysis reveals silver nanoparticle-decorated quercetin antibacterial molecular mechanism.” Acs Applied Materials & Interfaces 9, no. 11 (2017): 10047-10060.
Sydiskis R J, Owen D G, Lohr J L, Rosler K H, Blomster R N. Inactivation of enveloped viruses by anthraquinones extracted from plants. Antimicrob Agents Chemother. 1991; 35(12):2463-2466. doi:10.1128/aac.35.12.2463
Tahoori, Farnaz, M. A. J. D. Ahmad, Taher Nejadsattari, Hamideh Ofoghi, and Alireza Iranbakhsh. “Qualitative and Quantitative Study of Quercetin and Glycyrrhizin in In Vitro Culture of Liquorice (Glycyrrhiza glabra L.) and Elicitation with AgNO3.” Notulae Botanicae Horti Agrobotanici Cluj-Napoca 47, no. 1 (2019): 143-151.
Thacker P C, Karunagaran D. Curcumin and emodin down-regulate TGF-β signaling pathway in human cervical cancer cells. PLoS One. 2015; 10(3):e0120045. Published 2015 Mar. 18. doi:10.1371/journal.pone.0120045
Tiukavkina, et al., “Dihydroquercetin—new antioxidants and biologically active food additive”, Vopr Pitan 1997; (6):12-5
Tsuji M, Sriwilaijaroen N, Inoue H, et al. Synthesis and anti-influenza virus evaluation of triterpene-sialic acid conjugates. Bioorg Med Chem. 2018; 26(1):17-24. doi:10.1016/j.bmc.2017.09.038
Tyrsina E G, Rossikhina O G, Tyrsin IuA, Abilev SK.Dokl Akad Nauk, Ascorbyl palmitate—an antimutagen with membrane action]. SSSR. 1991; 318(4):992-4. Wang J, Chen X, Wang W, Zhang Y, Yang Z, Jin Y, Ge H M, Li E, Yang G. J Glycyrrhizic acid as the antiviral component of Glycyrrhiza uralensis Fisch. against coxsackievirus A16 and enterovirus 71 of hand foot and mouth disease. Ethnopharmacol. 2013 May 2; 147(1):114-21. doi: 10.1016/j.jep.2013.02.017. Epub 2013 Feb. 27
Uhari M, Kontiokari T, Koskela M, Niemela M. Xylitol chewing gum in prevention of acute otitis media: double blind randomized trial. BMJ. (1996 Nov. 9): 313(7066): 1180-1184. Uhari M, Kontiokari T, Niemela M. A Novel Use of Xylitol Sugar in Preventing Acute Otitis Media. Pediatrics (1998) 102(#4) (Oct. 4 1998) p. 879-884.
Uhari, Matti and Tero Kontiokari. U.S. Pat. No. 5,719,196. Feb. 15, 1998. Method of treating respiratory infections or complications derived therefrom in humans which includes oral administration of xylitol.
Welch J L, Xiang J, Okeoma C M, Schlievert P M, Stapleton J T. Glycerol Monolaurate, an Analogue to a Factor Secreted by Lactobacillus, Is Virucidal against Enveloped Viruses, Including HIV-1. mBio. 2020; 11(3):e00686-20. Published 2020 May 5. doi:10.1128/mBio.00686-20
Worth H, Dethlefsen U. Patients with asthma benefit from concomitant therapy with cineole: a placebo-controlled, double-blind trial. J Asthma. 2012; 49(8):849-853. doi:10.3109/02770903.2012.717657
Wu C C, Chen M S, Cheng Y J, et al. Emodin Inhibits EBV Reactivation and Represses NPC Tumorigenesis. Cancers (Basel). 2019; 11(11):1795. Published 2019 Nov. 15. doi:10.3390/cancers11111795
Wu, et al., “Synthesis and bio-activity of coumarin derivatives and studies on its relationships between activity and lipophilicity”, Yao Xue Xue Bao 1993; 28(4):266-72
Yang J K, Lin S S, Ji X J, Guo L M. Binding of SARS coronavirus to its receptor damages islets and causes acute diabetes. Acta Diabetol. 2010; 47(3):193-199. doi:10.1007/s00592-009-0109-4
Yu N, Sun Y T, Su X M, He M, Dai B, Kang J. Eucalyptol protects lungs against bacterial invasion through attenuating ciliated cell damage and suppressing MUC5AC expression. J Cell Physiol. 2019; 234(5):5842-5850. doi:10.1002/jcp.26359
Yu, Lumin, Fei Shang, Xiaolin Chen, Jingtian Ni, Li Yu, Ming Zhang, Dongdong Sun, and Ting Xue. “The anti-biofilm effect of silver-nanoparticle-decorated quercetin nanoparticles on a multi-drug resistant Escherichia coli strain isolated from a dairy cow with mastitis.” PeerJ6 (2018): e5711.
Zhang C H, Lifshitz L M, Uy K F, Ikebe M, Fogarty K E, ZhuGe R. The cellular and molecular basis of bitter tastant-induced bronchodilation [published correction appears in PLoS Biol. 2013 March; 11(3). doi:10.1371/annotation/7899a865-d68b-45bd-8b9b-ec6f50c9308a]. PLoS Biol. 2013; 11(3):e1001501. doi:10.1371/journal.pbio.1001501
Zhong T, Zhang L Y, Wang Z Y, et al. Rheum emodin inhibits enterovirus 71 viral replication and affects the host cell cycle environment. Acta Pharmacol Sin. 2017; 38(3):392-401. doi:10.1038/aps.2016.110
Zígolo M A, Salinas M, Alché L, Baldessari A, Linares G G. Chemoenzymatic synthesis of new derivatives of glycyrrhetinic acid with antiviral activity. Molecular docking study. Bioorg Chem. 2018; 78:210-219. doi:10.1016/j.bioorg.2018.03.018
US Patent and Pub. Patent Application Nos. RE46,698; 7,179,849; 7,378,156; 7,695,929; 7,985,557; 8,017,147; 8,034,454; 8,217,220; 8,389,021; 8,445,191; 8,486,620; 8,618,265; 8,703,490; 8,784,875; 8,846,320; 8,968,793; 8,968,794; 8,992,898; 9,017,954; 9,028,878; 9,040,090; 9,061,128; 9,103,822; 9,181,161; 9,408,393; 9,441,300; 9,492,364; 9,561,357; 9,566,223; 9,575,067; 9,597,676; 9,668,948; 9,719,986; 9,739,777; 9,902,818; 10,035,920; 10,071,037; 10,307,587; 10,314,931; 10,322,301; 10,329,299; 10,357,484; 10,357,669; 10,368,502; 10,375,952; 10,376,718; 10,391,060; 10,391,078; 10,392,371; 10,392,630; 10,398,673; 10,398,815; 10,406,174; 10,421,055; 10,434,052; 10,448,661; 10,449,348; 10,450,350; 10,463,613; 10,463,711; 10,465,188; 10,472,376; 10,480,007; 10,494,532; 10,519,188; 10,529,003; 10,538,475; 10,538,764; 10,544,181; 10,555,975; 10,561,633; 10,561,635; 10,568,931; 10,569,194; 10,577,315; 10,582,716; 10,583,086; 10,583,113; 10,588,957; 10,588,983; 10,596,146; 10,597,463; 10,610,488; 10,610,500; 10,619,167; 10,624,913; 10,627,412; 10,641,769; 10,653,717; 10,660,831; 10,675,286; 10,701,938; 10,718,693; 10,722,461; 10,731,046; 10,738,004; 10,743,604; 10,744,103; 10,760,075; 10,765,130; 10,772,854; 10,791,739; 10,799,450; 10,799,468; 10,806,707; 10,807,987; 10,813,355; 10,813,378; 10,821,506; 10,835,510; 10,849,867; 10,888,499; 10,888,618; 10,905,666; 10,912,746; 10,918,613; 10,919,914; 10,920,222; 10,933,011; 10,933,012; 10,933,017; 10,933,067; 10,941,162; 10,945,953; 10,947,206; 10,953,220; 10,960,012; 10,966,927; 10,968,231; 10,973,238; 10,980,756; 10,980,825; 10,987,250; 11,007,192; 11,013,685; 11,015,208; 11,053,262; 11,065,199; 11,076,539; 11,077,046; 11,077,068; 11,083,619; 11,084,855; 11,092,594; 20060147397; 20060165636; 20080260655; 20080292560; 20090202496; 20100011456; 20100041622; 20100221195; 20110296543; 20120266329; 20140134114; 20140178444; 20140248219; 20140363530; 20140378547; 20150173366; 20150240226; 20150250203; 20150367366; 20160023826; 20160058772; 20160060273; 20160067209; 20160081936; 20160095331; 20160113904; 20160115128; 20160136067; 20160136386; 20160136452; 20160145200; 20160151257; 20160152717; 20160158122; 20160175352; 20160177298; 20160184354; 20160192674; 20160199343; 20160201078; 20160206543; 20160206747; 20160219910; 20160222020; 20160228503; 20160228506; 20160235675; 20160235802; 20160237087; 20160243033; 20160244762; 20160271149; 20160272703; 20160286807; 20160310648; 20160317614; 20160319026; 20160326102; 20160326133; 20160326181; 20160331707; 20160338971; 20160339078; 20160339263; 20160347724; 20160347799; 20160354436; 20160361320; 20160361468; 20160367584; 20160367676; 20160369237; 20160374862; 20160375034; 20170000697; 20170000698; 20170000755; 20170015655; 20170020829; 20170028184; 20170035719; 20170042834; 20170049681; 20170049890; 20170056347; 20170056468; 20170058027; 20170064951; 20170065613; 20170065718; 20170066737; 20170079920; 20170080197; 20170080198; 20170080257; 20170095508; 20170100585; 20170105935; 20170105969; 20170106188; 20170107240; 20170114042; 20170119818; 20170135915; 20170135982; 20170143596; 20170150724; 20170157021; 20170164632; 20170166921; 20170172857; 20170173060; 20170182000; 20170189435; 20170189469; 20170204147; 20170208809; 20170216164; 20170216165; 20170217982; 20170224629; 20170224874; 20170226200; 20170234881; 20170246090; 20170246244; 20170246262; 20170253639; 20170258953; 20170273328; 20170275377; 20170283374; 20170283450; 20170290778; 20170296571; 20170298102; 20170304247; 20170316487; 20170327548; 20170333346; 20170349908; 20170360815; 20170360941; 20170361243; 20170362223; 20170368135; 20180002322; 20180002694; 20180008634; 20180028449; 20180028468; 20180042828; 20180042829; 20180042940; 20180045715; 20180055748; 20180055933; 20180071190; 20180071219; 20180078502; 20180084805; 20180092359; 20180099001; 20180105548; 20180117175; 20180118708; 20180120306; 20180125926; 20180162918; 20180185410; 20180208635; 20180222896; 20180230115; 20180230177; 20180231556; 20180235232; 20180237383; 20180243206; 20180256677; 20180263751; 20180271111; 20180273940; 20180289853; 20180291029; 20180296631; 20180303846; 20180305408; 20180311153; 20180318221; 20180319798; 20180325930; 20180326201; 20180343862; 20180344661; 20180353415; 20180360974; 20190000763; 20190008795; 20190008836; 20190010181; 20190016770; 20190029256; 20190038573; 20190038576; 20190038604; 20190054028; 20190060239; 20190062382; 20190075798; 20190076339; 20190076378; 20190083518; 20190090438; 20190091148; 20190099340; 20190105261; 20190142940; 20190151655; 20190169122; 20190192480; 20190200659; 20190201371; 20190223466; 20190224137; 20190224279; 20190225651; 20190233370; 20190233594; 20190240141; 20190240187; 20190247459; 20190255084; 20190275050; 20190290314; 20190290719; 20190290940; 20190298670; 20190298804; 20190300536; 20190308015; 20190320661; 20190328824; 20190329065; 20190350848; 20190358227; 20190366072; 20190374600; 20190380985; 20190388559; 20190389945; 20200000693; 20200002716; 20200010487; 20200010501; 20200022927; 20200022989; 20200024614; 20200030214; 20200030240; 20200031816; 20200031882; 20200039957; 20200048416; 20200060284; 20200060957; 20200060962; 20200069545; 20200069592; 20200071361; 20200078330; 20200085720; 20200087660; 20200093075; 20200093733; 20200095539; 20200101124; 20200109175; 20200113824; 20200115663; 20200138033; 20200147012; 20200147013; 20200154708; 20200170922; 20200171083; 20200179243; 20200179384; 20200188481; 20200197310; 20200197339; 20200197340; 20200197474; 20200208170; 20200222308; 20200222537; 20200224120; 20200224216; 20200231579; 20200231969; 20200239889; 20200246259; 20200246441; 20200247817; 20200247905; 20200254028; 20200263175; 20200268694; 20200276140; 20200277334; 20200281967; 20200289560; 20200297700; 20200306342; 20200316100; 20200317688; 20200330376; 20200330381; 20200339534; 20200345688; 20200354481; 20200354690; 20200377509; 20200385503; 20200390099; 20200390128; 20200397711; 20200397895; 20200397905; 20200405807; 20210009922; 20210009996; 20210015965; 20210017241; 20210037848; 20210037849; 20210038542; 20210046024; 20210052492; 20210052511; 20210052591; 20210052710; 20210059961; 20210061813; 20210068429; 20210069093; 20210069096; 20210085702; 20210092961; 20210114973; 20210115445; 20210121504; 20210121508; 20210127682; 20210138071; 20210139930; 20210161150; 20210177744; 20210179614; 20210180081; 20210198665; 20210198671; 20210205345; 20210205498; 20210212983; 20210213039; 20210213054; 20210213083; 20210214307; 20210219551; 20210220268; 20210228473; and 20210228684.

What is claimed is:

1. A pharmaceutically acceptable formulation comprising:
xylitol;
silver nanoparticles;
glycyrrhizin; and
quercetin.

2. The pharmaceutically acceptable formulation according to claim 1, further comprising: ascorbic acid; grapefruit seed extract; monolaurin; eucalyptol; emodin or aloe emodin; zinc citrate; and quinine.

3. The pharmaceutically acceptable formulation according to claim 1, wherein:
the pharmaceutically acceptable formulation is an oral or intranasal liquid;
the xylitol is present in an amount of at least 1 g/100 ml;
the silver nanoparticles are present in an amount of at least 0.001 g/100 ml;
the glycyrrhizin is present as glycyrrhizic acid in an amount of at least 0.1 g/100 ml; and
the quercetin is present in an amount of at least 0.1 g/100 ml.

4. The pharmaceutically acceptable formulation according to claim 1, wherein:
the pharmaceutically acceptable formulation is an oral or intranasal liquid;
the xylitol is present in an amount of between 1-10 g/100 ml;
the silver nanoparticles are present in an amount of between 0.001-0.020 g/100 ml;
the glycyrrhizin is present as glycyrrhizic acid in an amount of between 0.1-0.5 g/100 ml; and
the quercetin is present in an amount of between 0.1-5 g/100 ml.

5. The pharmaceutically acceptable formulation according to claim 4, further comprising ascorbic acid in an amount of between 0.1-3 g/100 ml.

6. The pharmaceutically acceptable formulation according to claim 4, further comprising grapefruit seed extract in an amount of between 0.1-0.5 g/100 ml.

7. The pharmaceutically acceptable formulation according to claim 4, further comprising monolaurin in an amount of between 0.1-0.5 g/100 ml.

8. The pharmaceutically acceptable formulation according to claim 4, further comprising eucalyptol in an amount of between 0.10-0.5 g/100 ml.

9. The pharmaceutically acceptable formulation according to claim 4, further comprising emodin in an amount of between 0.01-0.9 g/100 ml.

9. The pharmaceutically acceptable formulation according to claim 4, further comprising aloe emodin in an amount of between 0.01-0.9 g/100 ml.

10. The pharmaceutically acceptable formulation according to claim 4, further comprising zinc citrate in an amount of between 1-3 g/100 ml.

11. The pharmaceutically acceptable formulation according to claim 4, further comprising quinine in an amount of between 0.005-0.2 g/100 ml.

12. A method for reducing the transmissivity of an airborne bacteria or virus, comprising orally or intranasally administering to a subject a pharmaceutically acceptable formulation comprising xylitol; silver nanoparticles; glycyrrhizin; and quercetin.

13. The method according to claim 12, wherein the pharmaceutically acceptable formulation further comprises: ascorbic acid; grapefruit seed extract; monolaurin; eucalyptol; emodin or aloe emodin; zinc citrate; and quinine.

14. The method according to claim 12, wherein:
the pharmaceutically acceptable formulation is an intranasal liquid;
the xylitol is present in an amount of at least 1 g/100 ml;
the silver nanoparticles are present in an amount of at least 0.001 g/100 ml;
the glycyrrhizin is present as glycyrrhizic acid in an amount of at least 0.1 g/100 ml; and
the quercetin is present in an amount of at least 0.1 g/100 ml.

15. The method according to claim 14, wherein:
the xylitol is present in an amount of between 1-10 g/100 ml;
the silver nanoparticles are present in an amount of between 0.001-0.020 g/100 ml;
the glycyrrhizin is present as glycyrrhizic acid in an amount of between 0.1-0.5 g/100 ml; and
the quercetin is present in an amount of between 0.1-5 g/100 ml.

16. The method according to claim 12, further comprising determining an infection of the subject with the airborne bacteria or virus, and selectively intranasally administering the pharmaceutically acceptable formulation as a nasal spray in dependence on the determined infection.

17. The method according to claim 16, wherein the pathogen causing the determined infection is SARS-Cov2.

18. A pharmaceutical formulation in a light-proof multidose container, each dose comprising:
1-15 mg soluble oleoresin turmeric,
1-10% xylitol,
0.1-0.5% glycyrrhizic acid,
0.1-5% quercetin,
1-30 mg ascorbic acid,
0.1-0.5% grapefruit seed extract,
0.1-0.5% monolaurin,
0.01-0.5% eucalyptol,
0.01-0.9% emodin or aloe emodin, 1-3% zinc citrate,
5-200 ppm quinine,
at least one of potassium sorbate and benzalkonium chloride as a preservative, and
1-20% ethanol,
in an aqueous delivery system.

19. The pharmaceutical formulation according to claim 18, further comprising 1-20 μg silver nanoparticles.

20. The pharmaceutical formulation according to claim 19, further comprising thymol, a pharmaceutically acceptable flavoring agent, wherein the aqueous delivery system is adjusted to have a pH between pH 3.0 and pH 4.5.
